(19) United States
(12) Patent Application Publication
(10) Pub. No.: US 2015/0034874 A1
(43) Pub. Date: Feb. 5, 2015

(54) TRIFLUOROPHENYL DERIVATIVE, LIQUID CRYSTAL COMPOSITION, AND LIQUID CRYSTAL DISPLAY DEVICE

(71) Applicant: SEMICONDUCTOR ENERGY LABORATORY CO., LTD., Atsugi-shi (JP)

(72) Inventors: Momoko KATO, Iwaki (JP); Yuko KAWATA, Atsugi (JP); Yasuhiro NIIKURA, Zama (JP); Tetsuji ISHITANI, Atsugi (JP)

(21) Appl. No.: 14/341,863

(22) Filed: Jul. 28, 2014

(30) Foreign Application Priority Data
Jul. 31, 2013 (JP) 2013-159192

(51) Int. Cl. C09K 19/20; C09K 19/30; C07C 69/76; C09K 19/54

(52) U.S. Cl. CPC C09K 19/2007; C09K 19/54; C09K 19/3003; C07C 69/76; C09K 2019/3016; C09K 2019/2042; C09K 2019/2035
USPC 252/299.66; 252/299.67; 560/109; 560/102

(57) ABSTRACT

Provided is a novel material included in a liquid crystal composition that can be used in various liquid crystal display devices. A trifluorophenyl derivative represented by General Formula (G1). In General Formula (G1), Ar¹ represents an arylene group having 6 to 12 carbon atoms, a cycloalkylene group having 3 to 12 carbon atoms, or a cycloalkenylene group having 3 to 12 carbon atoms; m represents 1 or 2; and R¹ represents hydrogen, an alkyl group having 2 to 11 carbon atoms, or an alkoxy group having 2 to 11 carbon atoms.

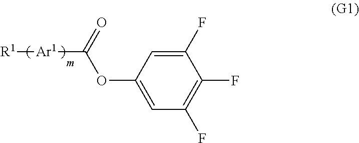

### BACKGROUND OF THE INVENTION

1. Field of the Invention

One embodiment of the present invention relates to a semiconductor device, a display device, a driving method thereof, or a manufacturing method thereof. Specifically, one embodiment of the present invention relates to a trifluorophenyl derivative, a liquid crystal composition, a liquid crystal display device, and manufacturing methods thereof.

2. Description of the Related Art

As a display device which is thin and lightweight (a flat panel display), a liquid crystal display device including a liquid crystal element, a light-emitting device including a self-light emitting element, a field emission display (an FED), and the like have been competitively developed.

In a liquid crystal display device, response speed of liquid crystal molecules is required to be increased. Among various kinds of display modes of liquid crystal, liquid crystal modes capable of high-speed response are a ferroelectric liquid crystal (FLC) mode, an optically compensated bend (OCB) mode, and a mode using liquid crystal exhibiting a blue phase.

In particular, the mode using liquid crystal exhibiting a blue phase does not need an alignment film and provides a wide viewing angle, and thus has been developed more actively for practical use (see Patent Documents 1 and 2, for example).

### REFERENCE

### Patent Document

[Patent Document 1] PCT International Publication No. 2005-090520
[Patent Document 2] Japanese Published Patent Application No. 2008-303381

### SUMMARY OF THE INVENTION

An object is to provide a liquid crystal composition exhibiting a blue phase, which enables higher contrast. Another object is to provide a trifluorophenyl derivative that can be used as a material for the liquid crystal composition. Another object is to provide a liquid crystal display device including the liquid crystal composition. Another object is to provide a novel liquid crystal composition.

Note that the descriptions of these objects do not disturb the existence of other objects. In one embodiment of the present invention, there is no need to achieve all the objects. Other objects will be apparent from and can be derived from the description of the specification, the drawings, the claims, and the like.

One embodiment of the invention disclosed in this specification is a trifluorophenyl derivative represented by General Formula (G1).

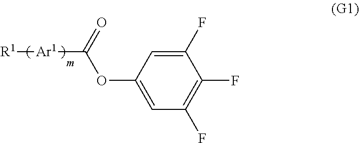

In General Formula (G1), Ar¹ represents an arylene group having 6 to 12 carbon atoms, a cycloalkylene group having 3 to 12 carbon atoms, or a cycloalkenylene group having 3 to 12 carbon atoms; m represents 1 or 2; and R¹ represents hydrogen, an alkyl group having 2 to 11 carbon atoms, or an alkoxy group having 2 to 11 carbon atoms.

Another embodiment of the invention disclosed in this specification is a trifluorophenyl derivative represented by General Formula (G2).

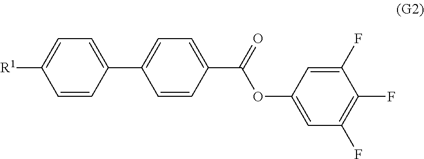

In General Formula (G2), R¹ represents hydrogen, an alkyl group having 2 to 11 carbon atoms, or an alkoxy group having 2 to 11 carbon atoms.

Another embodiment of the invention disclosed in this specification is a trifluorophenyl derivative represented by Structural Formula (103).

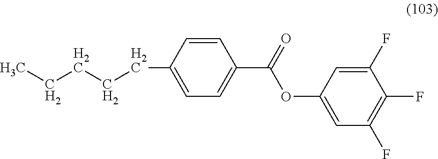 [SMILES: CCCCCc1ccc(C(=O)Oc2cc(F)c(F)c(F)c2)cc1]

Another embodiment of the invention disclosed in this specification is a trifluorophenyl derivative represented by Structural Formula (117).

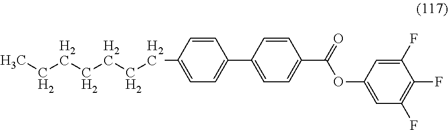 [SMILES: CCCCCCCc1ccc(-c2ccc(C(=O)Oc3cc(F)c(F)c(F)c3)cc2)cc1]

Another embodiment of the invention disclosed in this specification is a trifluorophenyl derivative represented by Structural Formula (119).

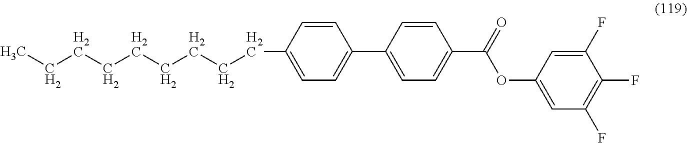 [SMILES: CCCCCCCCCc1ccc(-c2ccc(C(=O)Oc3cc(F)c(F)c(F)c3)cc2)cc1]

Another embodiment of the present invention is a liquid crystal composition including a chiral material and at least any one of trifluorophenyl derivatives represented by General Formula (G1), General Formula (G2), Structural Formula (103), Structural Formula (117), and Structural Formula (119).

A liquid crystal composition containing a chiral material can exhibit a blue phase by containing any one of the trifluorophenyl derivatives represented by General Formula (G1), General Formula (G2), Structural Formula (103), Structural Formula (117), and Structural Formula (119), each of which is one embodiment of the present invention.

A blue phase appears in a liquid crystal composition having strong twisting power and has a double twist structure. The liquid crystal composition shows a cholesteric phase, a cholesteric blue phase, an isotropic phase, or the like depending on conditions.

A cholesteric blue phase which is a blue phase includes three structures of blue phase I, blue phase II, and blue phase III from the low temperature side. A cholesteric blue phase which is a blue phase is optically isotropic, and blue phase I and blue phase II have body-centered cubic symmetry and simple cubic symmetry, respectively. In the cases of blue phase I and blue phase II, Bragg diffraction is seen in the range from ultraviolet light to visible light.

The chiral material is used to induce twisting of the liquid crystal composition, align the liquid crystal composition in a helical structure, and make the liquid crystal composition exhibit a blue phase. For the chiral material, a compound which has an asymmetric center, high compatibility with the liquid crystal composition, and strong twisting power is used. In addition, the chiral material is an optically active substance; a higher optical purity is better and the most preferable optical purity is 99% or higher.

When the twisting power of the liquid crystal composition is strong, the transmittance of the liquid crystal composition in the absence of an applied voltage (at an applied voltage of 0 V) can be low, leading to a higher contrast of a liquid crystal display device including the liquid crystal composition. Indicators of the strength of twisting power include the helical pitch, the selective reflection wavelength, HTP (helical twisting power), and the diffraction wavelength.

Addition of a large amount of chiral material increases voltage for driving the liquid crystal composition. However, the trifluorophenyl derivative contained in the liquid crystal composition has strong dielectric constant anisotropy owing to the action of the three fluorine atoms. Accordingly, the amount of chiral material can be small, and thus, driving voltage of a liquid crystal composition can be low in a blue phase liquid crystal mode; therefore, power consumption can be reduced.

A liquid crystal composition exhibiting a blue phase has an optical modulation property. It is optically isotropic at the time of no voltage application, whereas it becomes optically anisotropic when the alignment order changes by voltage application. The liquid crystal composition exhibiting a blue phase can be used for a liquid crystal display device.

A blue phase is optically isotropic and thus has no viewing angle dependence. Thus, an alignment film is not necessarily formed, which enables improvement in display image quality and cost reduction.

In a liquid crystal display device, it is preferable that a polymerizable monomer be added to a liquid crystal composition and polymer stabilization treatment be performed in order to broaden the temperature range within which a blue phase appears. As the polymerizable monomer, for example, a thermopolymerizable monomer which can be polymerized by heat, a photopolymerizable monomer which can be polymerized by light, or a polymerizable monomer which can be polymerized by heat and light can be used. Furthermore, a polymerization initiator may be added to the liquid crystal composition.

For example, polymer stabilization treatment can be performed in such a manner that a photopolymerizable monomer and a photopolymerization initiator are added to the liquid crystal composition and the liquid crystal composition is irradiated with light having a wavelength at which the photopolymerizable monomer and the photopolymerization initiator react with each other. When a UV-polymerizable monomer is used as a photopolymerizable monomer, the liquid crystal composition may be irradiated with ultraviolet light.

The liquid crystal composition exhibiting a blue phase is capable of high-speed response, which improves the performance of a liquid crystal display device. Note that the liquid crystal composition exhibiting a blue phase can be used in a mode other than a blue phase liquid crystal mode. Examples of the other modes are an optical rotation mode such as a TN mode or a cholesteric liquid crystal mode; a birefringence mode such as an IPS mode or an FFS mode; and a polymer dispersed liquid crystal mode.

One embodiment of the present invention includes, in its category, a liquid crystal element, a liquid crystal display device, and an electronic device each including any of the above liquid crystal compositions.

One embodiment of the present invention can provide a liquid crystal composition exhibiting a blue phase, which achieves high contrast. Another embodiment of the present invention can provide a trifluorophenyl derivative that can be used as a material of the liquid crystal composition. Another embodiment of the present invention can provide a liquid crystal display device including the liquid crystal composition. Another embodiment of the present invention can provide a novel liquid crystal composition.

### BRIEF DESCRIPTION OF THE DRAWINGS

FIG. 1 is a conceptual view illustrating a liquid crystal composition.

FIGS. 2A and 2B illustrate one embodiment of a liquid crystal display device.

FIGS. 3A to 3D each illustrate one embodiment of an electrode structure of a liquid crystal display device.

FIGS. 4A and 4B illustrate one embodiment of a liquid crystal display device.

FIGS. 5A to 5D each illustrate one embodiment of an electrode structure of a liquid crystal display device.

FIGS. 6A and 6B illustrate one embodiment of a liquid crystal display device.

FIGS. 7A1, 7A2, and 7B illustrate liquid crystal display modules.

FIGS. 8A and 8B illustrate an electronic device.

FIGS. 9A to 9F illustrate electronic devices.

FIGS. 10A and 10B are ¹H NMR charts of PEP-5FFF.

FIG. 11 is a ¹H NMR chart of CPP-PEP-5FFF.

FIGS. 12A and 12B are ¹H NMR charts of PPEP-7FFF.

FIG. 13 is a ¹H NMR chart of PPEP-7FFF.

FIGS. 14A and 14B are ¹H NMR charts of PPEP-9FFF.

FIG. 15 is a ¹H NMR chart of PPEP-9FFF.

### DETAILED DESCRIPTION OF THE INVENTION

Embodiments and examples will be described in detail with reference to the accompanying drawings. Note that the present invention is not limited to the description below, and it is easily understood by those skilled in the art that a variety of changes and modifications can be made without departing from the spirit and scope of the present invention. Therefore, the present invention should not be construed as being limited to the descriptions of the embodiments and the examples below. In the structures to be given below, the same portions or portions having similar functions are denoted by the same reference numerals in different drawings, and descriptions thereof will not be repeated.

Note that the ordinal numbers such as “first”, “second”, and “third” in this specification are used for convenience and do not denote the order of steps and the stacking order of layers. Therefore, for example, the term “first” can be replaced with the term “second”, “third”, or the like as appropriate. In addition, the ordinal numbers in this specification and the like are not necessarily the same as the ordinal numbers used to specify one embodiment of the present invention.

In this specification, a semiconductor device means a general device that can function by utilizing semiconductor characteristics, and an electrooptic device, a semiconductor circuit, and an electronic device are all semiconductor devices.

### Embodiment 1

In this embodiment, a trifluorophenyl derivative of one embodiment of the present invention will be described.

The trifluorophenyl derivative of this embodiment is represented by General Formula (G1).

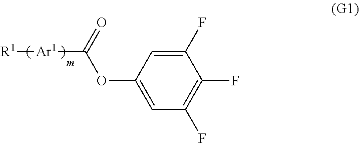

In General Formula (G1), Ar¹ represents an arylene group having 6 to 12 carbon atoms, a cycloalkylene group having 3 to 12 carbon atoms, or a cycloalkenylene group having 3 to 12 carbon atoms; m represents 1 or 2; and R¹ represents hydrogen, an alkyl group having 2 to 11 carbon atoms, or an alkoxy group having 2 to 11 carbon atoms.

Specifically, the trifluorophenyl derivative of this embodiment is represented by General Formula (G2).

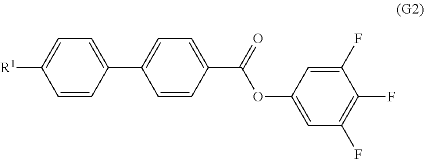

In General Formula (G2), R¹ represents hydrogen, an alkyl group having 2 to 11 carbon atoms, or an alkoxy group having 2 to 11 carbon atoms.

Specific examples of the trifluorophenyl derivatives represented by General Formulae (G1) and (G2) are trifluorophenyl derivatives represented by Structural Formulae (100) to (123). Note that one embodiment of the present invention is not limited to these examples.

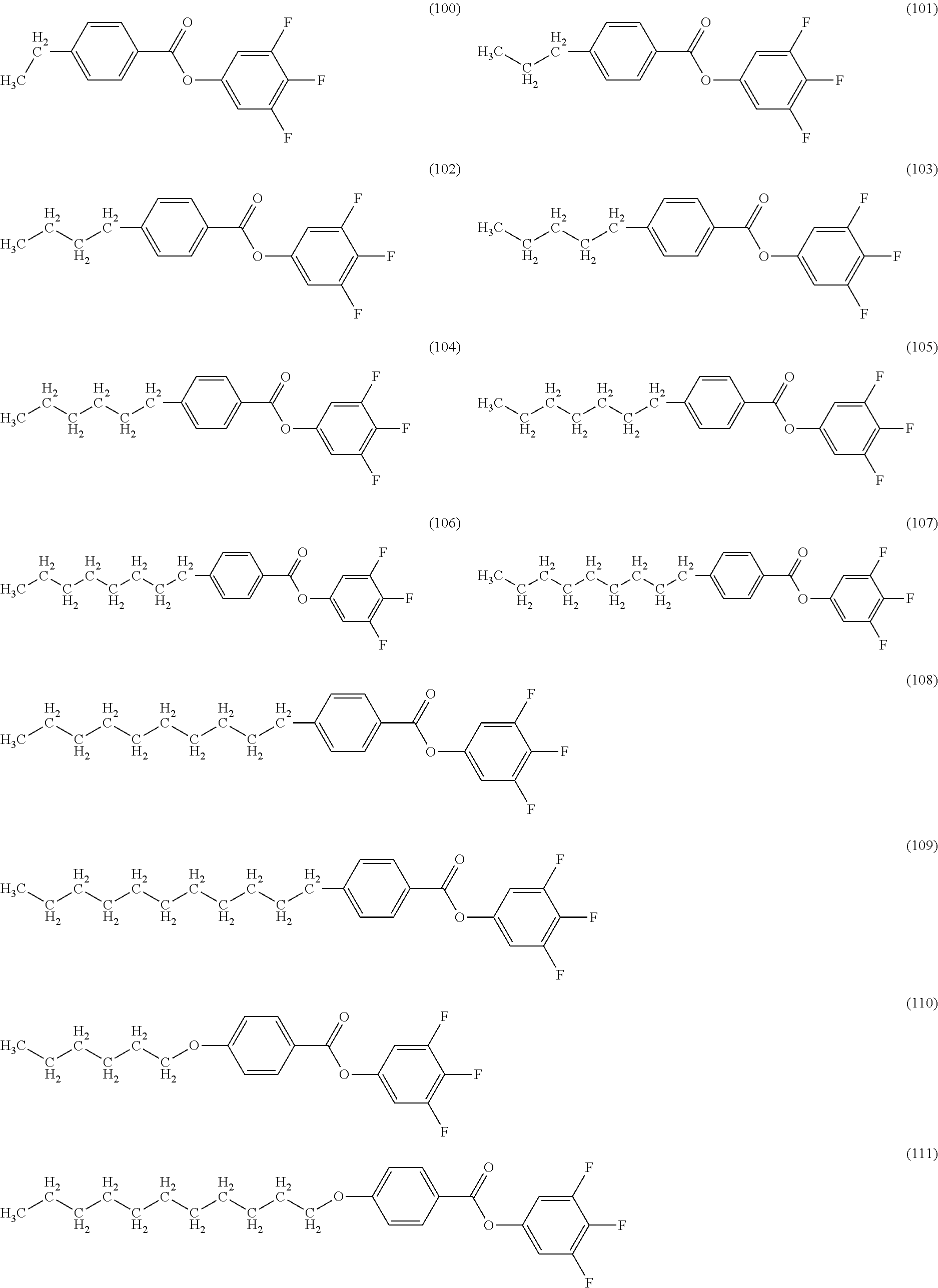

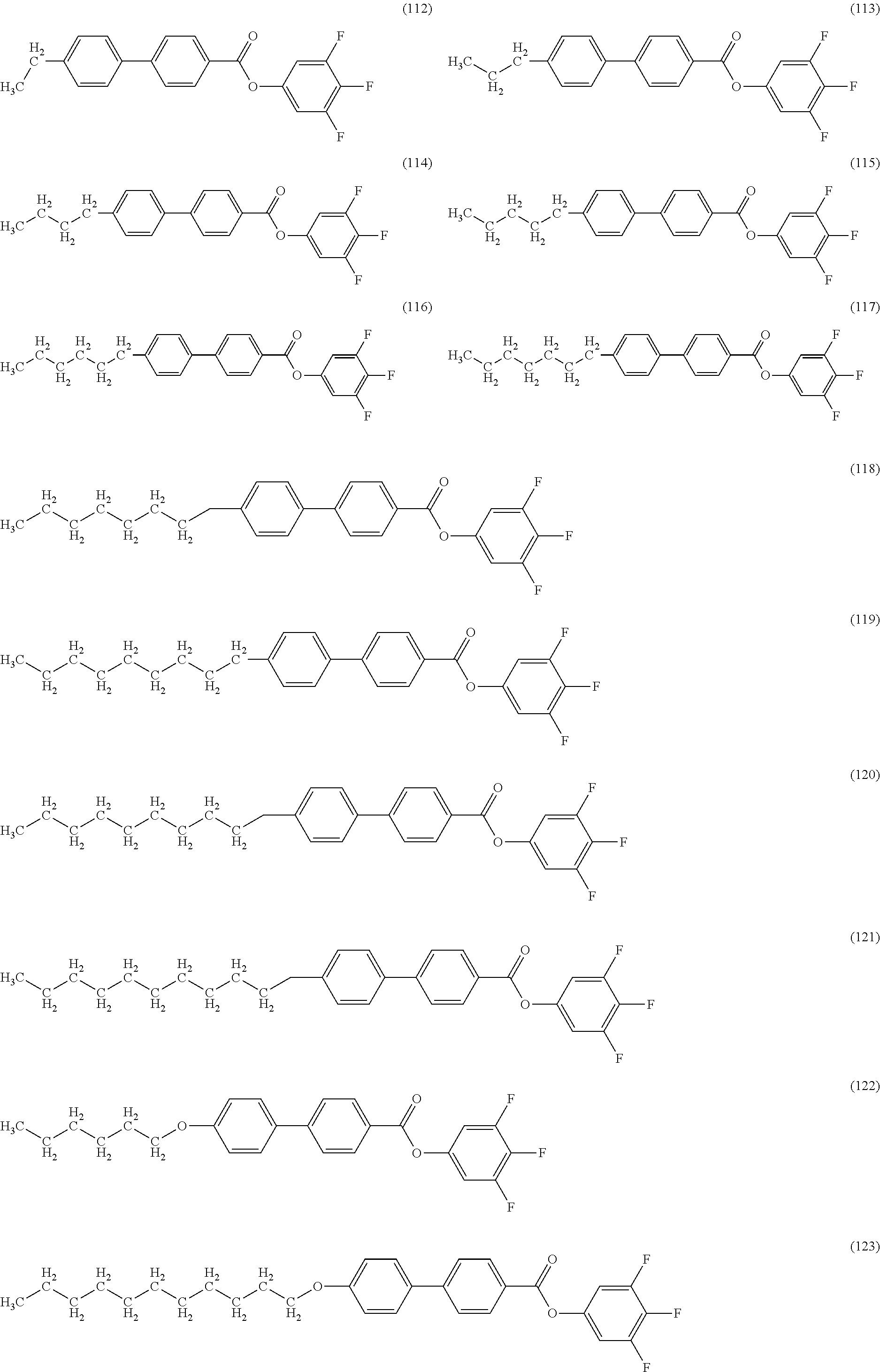

A variety of reactions can be applied to a method of synthesizing the trifluorophenyl derivative of this embodiment. The following is an example of a method of synthesizing the trifluorophenyl derivative represented by General Formula (G1).

The trifluorophenyl derivative represented by General Formula (G1) can be synthesized by the reaction represented by the following Scheme (K-1).

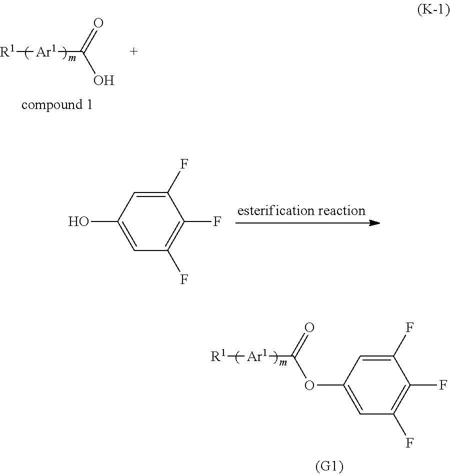

By an esterification reaction of Compound 1 with 3,4,5-trifluorophenol, the trifluorophenyl derivative represented by General Formula (G1) can be obtained (Scheme (K-1)).

In General Formula (G1), Ar¹ represents an arylene group having 6 to 12 carbon atoms, a cycloalkylene group having 3 to 12 carbon atoms, or a cycloalkenylene group having 3 to 12 carbon atoms; m represents 1 or 2; and R¹ represents hydrogen, an alkyl group having 2 to 11 carbon atoms, or an alkoxy group having 2 to 11 carbon atoms.

The following is an example of a method of synthesizing the trifluorophenyl derivative represented by General Formula (G2). The trifluorophenyl derivative represented by General Formula (G2) can be synthesized by the reaction represented by the following Scheme (K2-1).

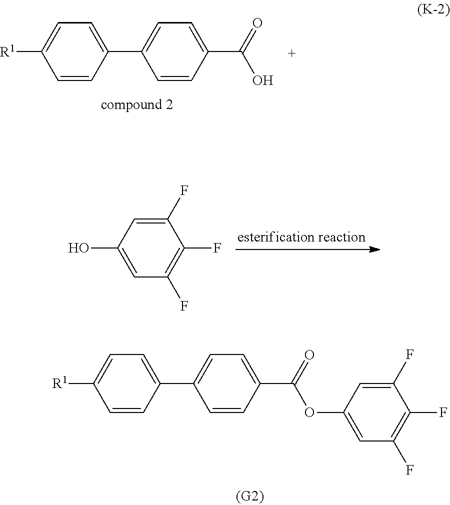

By an esterification reaction of Compound 2 with 3,4,5-trifluorophenol, the trifluorophenyl derivative represented by General Formula (G2) can be obtained (Scheme (K-2)).

In General Formula (G2), R¹ represents hydrogen, an alkyl group having 2 to 11 carbon atoms, or an alkoxy group having 2 to 11 carbon atoms. In General Formulae (G1) and (G2), an arylene group having 6 to 12 carbon atoms, a cycloalkylene group having 3 to 12 carbon atoms, a cycloalkenylene group having 3 to 12 carbon atoms, an alkyl group having 2 to 11 carbon atoms, or an alkoxy group having 2 to 11 carbon atoms may have a substituent. Examples of the substituent include fluorine (F), chlorine (Cl), bromine (Br), iodine (I), a cyano group (CN), a trifluoromethylsulfonyl group (SO₂CF₃), a trifluoromethyl group (CF₃), a nitro group (NO₂), an isothiocyanate group (NCS), and a pentafluorosulfanyl group (SF₅).

In the above manner, the trifluorophenyl derivative of one embodiment of the present invention can be synthesized.

The chiral material is used to induce twisting of the liquid crystal composition, align the liquid crystal composition in a helical structure, and make the liquid crystal composition exhibit a blue phase. For the chiral material, a compound which has an asymmetric center, high compatibility with the liquid crystal composition, and strong twisting power is used. In addition, the chiral material is an optically active substance; a higher optical purity is better and the most preferable optical purity is 99% or higher.

The liquid crystal composition exhibiting a blue phase, which is disclosed in this specification, can be used for a liquid crystal display device.

A blue phase is optically isotropic and thus has no viewing angle dependence. Thus, an alignment film is not necessarily formed, which enables improvement in display image quality and cost reduction.

In the case where the liquid crystal composition described in this embodiment exhibits a blue phase, it is preferable that a polymerizable monomer be added to a liquid crystal composition and polymer stabilization treatment be performed in order to broaden the temperature range within which a blue phase is exhibited in a liquid crystal display device. As the polymerizable monomer, for example, a thermopolymerizable (thermosetting) oligomers which can be polymerized by heat, a photopolymerizable (photocurable) oligomers which can be polymerized by light, and the like can be used in addition to vinyl monomers.

The polymerizable vinyl monomer may be a monofunctional monomer such as an acrylate or a methacrylate; a polyfunctional monomer such as a diacrylate, a triacrylate, a dimethacrylate, or a trimethacrylate; or a mixture thereof. The polymerizable monomer may have liquid crystallinity, non-liquid crystallinity, or a mixture of them.

A polymerization initiator may be added to the liquid crystal composition when the polymer stabilization treatment is carried out. As the polymerization initiator, a radical polymerization initiator which generates radicals by light irradiation or heating, an acid generator which generates an acid by light irradiation or heating, or a base generator which generates a base by light irradiation or heating may be used.

For example, polymer stabilization treatment can be performed in such a manner that a polymerizable monomer and a photopolymerization initiator are added to the liquid crystal composition and the liquid crystal composition is irradiated with light.

This polymer stabilization treatment may be performed in a state that a liquid crystal composition exhibits an isotropic phase or in a state that a liquid crystal composition exhibits a blue phase under the temperature control. A temperature at which the phase changes from a blue phase to an isotropic phase when the temperature is increased, or a temperature at which the phase changes from an isotropic phase to a blue phase when the temperature is decreased is referred to as the phase transition temperature between a blue phase and an isotropic phase. For example, the polymer stabilization treatment can be performed in the following manner: after a liquid crystal composition to which a photopolymerizable monomer is added is heated to exhibit an isotropic phase, the temperature of the liquid crystal composition is gradually decreased until the phase changes to a blue phase, and then, light irradiation is performed while the temperature at which a blue phase is exhibited is kept.

### Embodiment 2

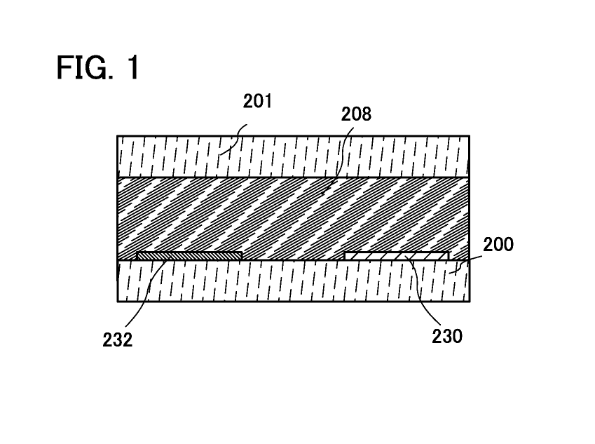

A liquid crystal composition of one embodiment of the structure of the invention disclosed in this specification, and a liquid crystal display device including the liquid crystal composition will be described with reference to FIG. 1. FIG. 1 illustrates a cross section of a liquid crystal display device in which a first substrate 200 and a second substrate 201 are positioned so as to face each other with a liquid crystal composition 208 exhibiting a blue phase interposed between the first substrate 200 and the second substrate 201. A pixel electrode layer 230 and a common electrode layer 232 are provided between the first substrate 200 and the liquid crystal composition 208 so as to be adjacent to each other.

In a liquid crystal display device including a liquid crystal composition exhibiting a blue phase, a method can be used in which the gray scale is controlled by moving liquid crystal molecules in a plane parallel to the substrate with the application of an electric field parallel to or substantially parallel to a substrate (i.e., in the lateral direction).

The maximum thickness (film thickness) of the liquid crystal composition 208 is preferably greater than or equal to 1 μm and less than or equal to 20 μm.

The liquid crystal composition 208 can be formed by a dispenser method (a dropping method), or an injection method by which liquid crystal is injected using capillary action or the like after the first substrate 200 and the second substrate 201 are attached to each other.

A liquid crystal composition including a trifluorophenyl derivative and a chiral material is used as the liquid crystal composition 208. The liquid crystal composition provided as the liquid crystal composition 208 may further include an organic resin.

With an electric field generated between the pixel electrode layer 230 and the common electrode layer 232, liquid crystal is controlled. An electric field in the lateral direction is generated in the liquid crystal, so that liquid crystal molecules can be controlled using the electric field.

The liquid crystal composition exhibiting a blue phase is capable of high-speed response. Thus, a high-performance liquid crystal display device can be achieved.

For example, the quick response of such a liquid crystal composition exhibiting a blue phase allows the application of a successive additive color mixing method (a field sequential method) or a three-dimensional display method. In the successive additive color mixing method, light-emitting diodes (LEDs) of RGB or the like are arranged in a backlight unit and color display is performed by time division. In the three-dimensional display method, a shutter glasses system is used in which images for a right eye and images for a left eye are alternately viewed by time division.

Although not illustrated in FIG. 1, an optical film such as a polarizing plate, a retardation plate, an anti-reflection film, or the like may be provided as appropriate. For example, circular polarization by the polarizing plate and the retardation plate may be used. In addition, a backlight or the like can be used as a light source.

In this specification, the first substrate can be provided with a semiconductor element (e.g., a transistor), a pixel electrode layer, and a common electrode layer. In this case, the first substrate is also referred to as an element substrate, and the second substrate which faces the element substrate with a liquid crystal composition interposed therebetween is also referred to as a counter substrate.

With the use of the liquid crystal composition exhibiting a blue phase, which is disclosed in this specification, for a liquid crystal display device, a transmissive liquid crystal display device in which display is performed by transmission of light from a light source, a reflective liquid crystal display device in which display is performed by reflection of incident light, or a transflective liquid crystal display device in which a transmissive type and a reflective type are combined can be provided.

In the case of the transmissive liquid crystal display device, a first substrate, a second substrate, and other components such as an insulating film and a conductive film, which are provided in a light-transmitting pixel region, have a light-transmitting property. It is preferable that the pixel electrode layer and the common electrode layer have a light-transmitting property; however, if an opening is provided, a non-light-transmitting material such as a metal film may be used depending on the shape.

On the other hand, in the case of the reflective liquid crystal display device, a reflective component which reflects light transmitted through the liquid crystal composition (e.g., a reflective film or substrate) may be provided on the side opposite to the viewing side of the liquid crystal composition. Therefore, a substrate, an insulating film, and a conductive film which are provided between the viewing side and the reflective component and through which light is transmitted have a light-transmitting property. Note that in this specification, a light-transmitting property refers to a property of transmitting at least visible light.

The pixel electrode layer 230 and the common electrode layer 232 may be formed with the use of one or more of the following: indium tin oxide (ITO); a conductive material in which zinc oxide (ZnO) is mixed into indium oxide; a conductive material in which silicon oxide (SiO₂) is mixed into indium oxide; indium oxide containing tungsten oxide; indium zinc oxide containing tungsten oxide; indium oxide containing titanium oxide; and indium tin oxide containing titanium oxide; graphene; metals such as tungsten (W), molybdenum (Mo), zirconium (Zr), hafnium (Hf), vanadium (V), niobium (Nb), tantalum (Ta), chromium (Cr), cobalt (Co), nickel (Ni), titanium (Ti), platinum (Pt), aluminum (Al), copper (Cu), and silver (Ag); alloys thereof; and metal nitrides thereof.

As the first substrate 200 and the second substrate 201, a glass substrate of barium borosilicate glass, aluminoborosilicate glass, or the like, a quartz substrate, a plastic substrate, or the like can be used.

This embodiment can be implemented in an appropriate combination with any of the structures described in the other embodiments.

### Embodiment 3

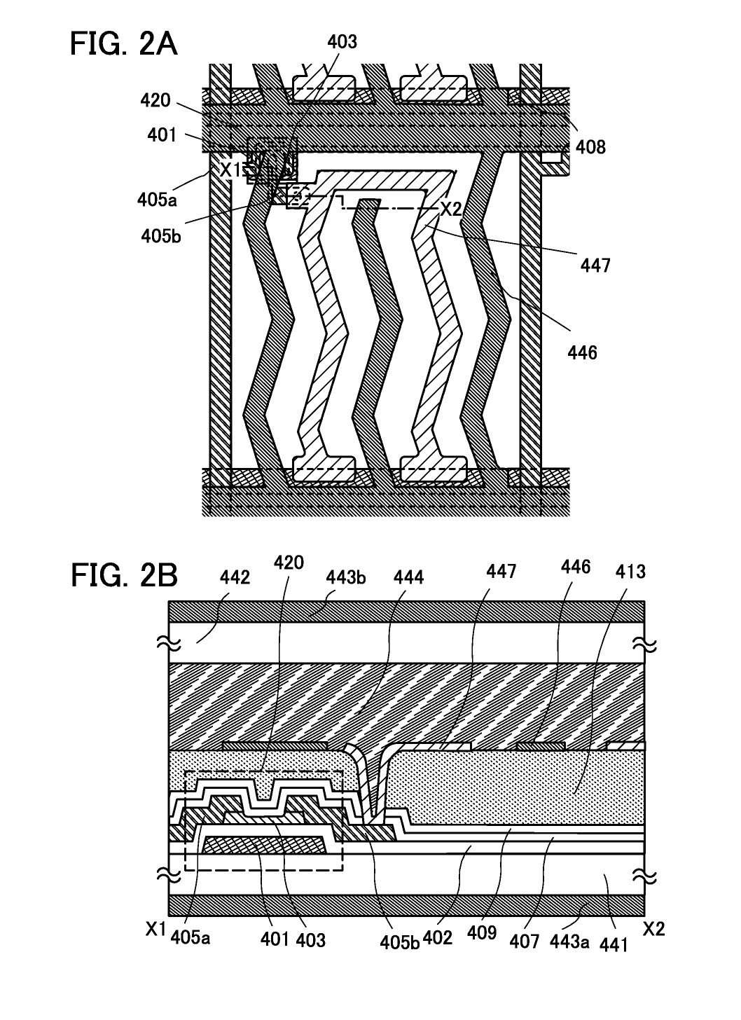

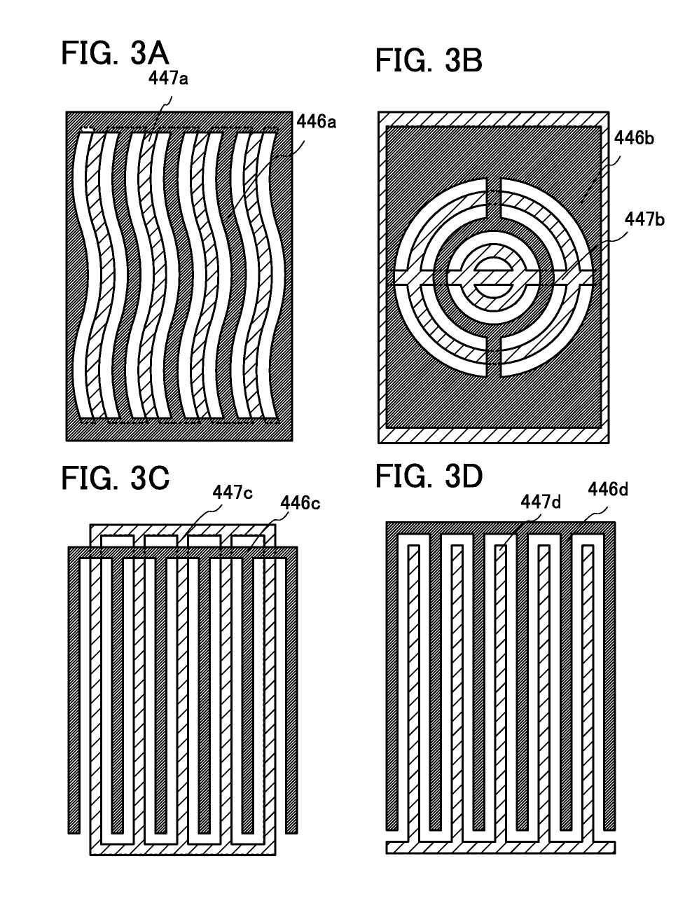

The invention disclosed in this specification can be applied to both a passive matrix liquid crystal display device and an active matrix liquid crystal display device. In this embodiment, an example of an active matrix liquid crystal display device to which the invention disclosed in this specification is applied will be described with reference to FIGS. 2A and 2B and FIGS. 3A and 3D.

FIG. 2A is a plan view of the liquid crystal display device and illustrates one pixel. FIG. 2B is a cross-sectional view taken along line X1-X2 in FIG. 2A.

In FIG. 2A, a plurality of source wiring layers (including a wiring layer 405a) is arranged so as to be parallel to (extend in the vertical direction in the drawing) and apart from each other. A plurality of gate wiring layers (including a gate electrode layer 401) is provided to extend in a direction substantially perpendicular to the source wiring layers (the horizontal direction in the drawing) and to be apart from each other. Common wiring layers 408 are provided adjacent to the respective plurality of gate wiring layers and extend in a direction substantially parallel to the gate wiring layers, that is, in a direction substantially perpendicular to the source wiring layers (the horizontal direction in the drawing). A roughly rectangular space is surrounded by the source wiring layers, the common wiring layers 408, and the gate wiring layers. In this space, a pixel electrode layer and a common electrode layer of the liquid crystal display device are provided. A transistor 420 for driving the pixel electrode layer is provided at an upper left corner of the drawing. A plurality of pixel electrode layers and a plurality of transistors are arranged in matrix.

In the liquid crystal display device of FIGS. 2A and 2B, a first electrode layer 447 which is electrically connected to the transistor 420 serves as a pixel electrode layer, while a second electrode layer 446 which is electrically connected to the common wiring layer 408 serves as a common electrode layer. Note that a capacitor is formed by the first electrode layer and the common wiring layer. Although the common electrode layer can operate in a floating state (an electrically isolated state), the potential of the common electrode layer may be set to a fixed potential, preferably to a potential around a common potential (an intermediate potential of an image signal which is transmitted as data) in such a level as not to generate flickers.

It is possible to employ a driving method in which the gray scale is controlled by generating an electric field substantially parallel (i.e., in a lateral direction) to a substrate to orientate liquid crystal molecules in a plane parallel to the substrate. For such a method, an electrode structure used in an IPS mode as illustrated in FIGS. 2A and 2B and FIGS. 3A to 3D can be employed.

In a lateral electric field mode such as an IPS mode, a first electrode layer (e.g., a pixel electrode layer a voltage of which is controlled in each pixel) and a second electrode layer (e.g., a common electrode layer to which a common voltage is supplied in all pixels), each of which has an opening pattern, are located below a liquid crystal composition, for example. Therefore, the first electrode layer 447 and the second electrode layer 446, one of which is a pixel electrode layer and the other is a common electrode layer, are formed over a first substrate 441, and at least one of the first electrode layer and the second electrode layer is formed over an interlayer film. The first electrode layer 447 and the second electrode layer 446 have a variety of shapes. For example, they can have an opening portion, a bent portion, branched portion, or a comb-shaped portion. In order to generate an electric field substantially parallel to a substrate between the first electrode layer 447 and the second electrode layer 446, an arrangement is avoided in which they have the same shape and completely overlap with each other.

As the liquid crystal composition 444, the liquid crystal composition exhibiting a blue phase and including the trifluorophenyl derivative shown in Embodiment 1 and a chiral material is used. The liquid crystal composition 444 may further include an organic resin. In this embodiment, the liquid crystal composition 444 is formed by performing the polymer stabilization treatment in a state that the liquid crystal composition 444 exists in a blue phase.

With a lateral electric field generated between the first electrode layer 447 and the second electrode layer 446, liquid crystal of the liquid crystal composition 444 is controlled. Hence, a wide viewing angle can be obtained.

FIGS. 3A to 3D show other examples of the first electrode layer 447 and the second electrode layer 446. As illustrated in top views of FIGS. 3A to 3D, first electrode layers 447a to 447d and second electrode layers 446a to 446d are staggered. In FIG. 3A, the first electrode layer 447a and the second electrode layer 446a have an opening with a wavelike shape. In FIG. 3B, the first electrode layer 447b and the second electrode layer 446b have a shape with concentric circular openings. In FIG. 3C, the first electrode layer 447c and the second electrode layer 446c have a comb-shape and partially overlap with each other. In FIG. 3D, the first electrode layer 447d and the second electrode layer 446d have a comb-shape in which the electrode layers are engaged with each other. In the case where the first electrode layers 447a, 447b, and 447c overlap with the second electrode layers 446a, 446b, and 446c, respectively, as illustrated in FIGS. 3A to 3C, an insulating film is formed between the first electrode layer 447 and the second electrode layer 446 so that the first electrode layer 447 and the second electrode layer 446 are formed over different films.

Since the second electrode layer 446 has an opening pattern, they are illustrated as a divided plurality of electrode layers in the cross-sectional view of FIG. 2B. The same applies to the other drawings of this specification.

The transistor 420 is an inverted staggered thin film transistor in which the gate electrode layer 401, a gate insulating layer 402, a semiconductor layer 403, and wiring layers 405a and 405b which function as a source electrode layer and a drain electrode layer are formed over the first substrate 441 which has an insulating surface.

There is no particular limitation on a structure of a transistor that can be used for a liquid crystal display device disclosed in this specification. For example, a staggered type or a planar type having a top-gate structure or a bottom-gate structure can be employed. The transistor may have a single-gate structure in which one channel formation region is formed, a double-gate structure in which two channel formation regions are formed, or a triple-gate structure in which three channel formation regions are formed. Alternatively, the transistor may have a dual gate structure including two gate electrode layers positioned over and below a channel region with a gate insulating layer provided therebetween.

An insulating film 407 that is in contact with the semiconductor layer 403, and an insulating film 409 are provided to cover the transistor 420. An interlayer film 413 is stacked over the insulating film 409.

There is no particular limitation on the method of forming the interlayer film 413, and the following method can be employed depending on the material: spin coating, dip coating, spray coating, a droplet discharging method (e.g., an ink-jetting), a printing method (e.g., screen printing method or offset printing), roll coating, curtain coating, knife coating, or the like.

The first substrate 441 and a second substrate 442 that is a counter substrate are firmly attached to each other with a sealant with the liquid crystal composition 444 interposed therebetween. The liquid crystal composition 444 can be formed by a dispenser method (a dropping method), or an injection method by which the liquid crystal composition 444 is injected using capillary action or the like after the first substrate 441 is attached to the second substrate 442.

As the sealant, typically, a visible light curable resin, a UV curable resin, or a thermosetting resin is preferably used. Typically, an acrylic resin, an epoxy resin, an amine resin, or the like can be used. Further, a filler or a coupling agent may be included in the sealant.

In this embodiment, polymer stabilization treatment is performed on the liquid crystal composition 444 by adding a photopolymerizable monomer and a photopolymerization initiator to the liquid crystal composition exhibiting a blue phase and including a chiral material and the trifluorophenyl derivative shown in Embodiment 1.

After the space between the first substrate 441 and the second substrate 442 is filled with the liquid crystal composition, polymer stabilization treatment is performed by light irradiation, whereby the liquid crystal composition 444 is formed. The light has a wavelength with which the polymerizable monomer and the photopolymerization initiator which are contained in the liquid crystal composition used as the liquid crystal composition 444 react with each other. By such polymer stabilization treatment by light irradiation, the temperature range within which the liquid crystal composition 444 exhibits a blue phase can be broadened.

The liquid crystal composition according to this embodiment has strong twisting power, and in the liquid crystal composition 444 subjected to polymer stabilization treatment, the peak of the diffracted wavelength on the longest wavelength side in the reflectance spectrum can be a short wavelength (preferably, less than or equal to 450 nm, more preferably less than or equal to 420 nm). Thus, the transmittance of the liquid crystal composition in the absence of an applied voltage (at an applied voltage of 0 V) can be low, leading to a higher contrast ratio of a liquid crystal display device.

In the case where a photocurable resin such as a UV curable resin is used as a sealant and a liquid crystal composition is formed by a dropping method, for example, the sealant may be cured in the light irradiation step of the polymer stabilization treatment.

In this embodiment, a polarizing plate 443a is provided on the outer side (on the side opposite to the liquid crystal composition 444) of the first substrate 441, and a polarizing plate 443b is provided on the outer side (on the side opposite to the liquid crystal composition 444) of the second substrate 442. In addition to the polarizing plates, an optical film such as a retardation plate or an anti-reflection film may be provided. For example, circular polarization plate formed by the polarizing plate and the retardation plate may be used. Through the above-described process, a liquid crystal display device is completed.

In the case of manufacturing a plurality of liquid crystal display devices using a large-sized substrate (a multiple panel method), a division step can be performed before the polymer stabilization treatment is performed or before the polarizing plates are provided. In consideration of the influence of the division step on the liquid crystal composition (such as alignment disorder due to force applied in the division step), it is preferable that the division step be performed after the attachment between the first substrate and the second substrate and before the polymer stabilization treatment.

Although not illustrated, a backlight, a sidelight, or the like may be used as a light source. Light source is provided so that light passes the second substrate 442 (the viewing side) through the first substrate 441 (the element substrate).

The first electrode layer 447 and the second electrode layer 446 can be formed using a light-transmitting conductive material such as indium oxide containing tungsten oxide, indium zinc oxide containing tungsten oxide, indium oxide containing titanium oxide, indium tin oxide containing titanium oxide, indium tin oxide, indium zinc oxide, indium tin oxide to which silicon oxide is added, or graphene.

The first electrode layer 447 and the second electrode layer 446 can be formed using one or more kinds selected from a metal such as tungsten (W), molybdenum (Mo), zirconium (Zr), hafnium (Hf), vanadium (V), niobium (Nb), tantalum (Ta), chromium (Cr), cobalt (Co), nickel (Ni), titanium (Ti), platinum (Pt), aluminum (Al), copper (Cu), or silver (Ag); an alloy thereof; and a nitride thereof.

The first electrode layer 447 and the second electrode layer 446 can be formed with the use of a conductive composition including a conductive macromolecule (also referred to as conductive polymer). The pixel electrode formed with the use of the conductive composition preferably has a sheet resistance of less than or equal to 10000 ohms per square and a transmittance of greater than or equal to 70% at a wavelength of 550 nm. Furthermore, the resistivity of the conductive macromolecule included in the conductive composition is preferably less than or equal to 0.1 Ω·cm.

As the conductive polymer, what is called a π-conjugated conductive polymer can be used. Examples include polyaniline or a derivative thereof, polypyrrole or a derivative thereof, polythiophene or a derivative thereof, and a copolymer of two or more of aniline, pyrrole, and thiophene or a derivative thereof.

An insulating film serving as a base film may be provided between the first substrate 441 and the gate electrode layer 401. The base film has a function of preventing diffusion of an impurity element from the first substrate 441, and can be formed to have a single-layer or layered structure using one or more of a silicon nitride film, a silicon oxide film, a silicon nitride oxide film, and a silicon oxynitride film. The gate electrode layer 401 can be a single-layer or layered structure using a metal such as molybdenum, titanium, chromium, tantalum, tungsten, aluminum, copper, neodymium, or scandium, or an alloy which contains any of these metals as its main component. By using a light-blocking conductive film as the gate electrode layer 401, light from a backlight (light passing through the first substrate 441) can be prevented from entering the semiconductor layer 403.

For example, as a two-layer structure of the gate electrode layer 401, the following structures are preferable: a two-layer structure in which a molybdenum layer is stacked over an aluminum layer, a two-layer structure in which a molybdenum layer is stacked over a copper layer, a two-layer structure in which a titanium nitride layer or a tantalum nitride layer is stacked over a copper layer, and a two-layer structure in which a titanium nitride layer and a molybdenum layer are stacked. As a three-layer structure, a layered structure in which a tungsten layer or a tungsten nitride layer, an alloy layer of aluminum and silicon or an alloy layer of aluminum and titanium, and a titanium nitride layer or a titanium layer are stacked is preferable.

The gate insulating layer 402 can be formed using a silicon oxide layer, a silicon nitride layer, a silicon oxynitride layer, or a silicon nitride oxide layer to have a single-layer or stacked-layer structure by a plasma CVD method, a sputtering method, or the like. Note that the silicon oxide layer can be formed by a CVD method in which an organosilane gas is used. As an organosilane gas, a silicon-containing compound such as tetraethoxysilane (TEOS) (chemical formula: Si(OC₂H₅)₄), tetramethylsilane (TMS) (chemical formula: Si(CH₃)₄), tetramethylcyclotetrasiloxane (TMCTS), octamethylcyclotetrasiloxane (OMCTS), hexamethyldisilazane (HMDS), triethoxysilane (chemical formula: SiH(OC₂H₅)₃), or trisdimethylaminosilane (chemical formula: SiH(N(CH₃)₂)₃) can be used.

A material of the semiconductor layer 403 is not limited to a particular material and may be determined in accordance with characteristics needed for the transistor 420, as appropriate. Examples of a material that can be used for the semiconductor layer 403 will be described.

The semiconductor layer 403 can be formed using the following material: an amorphous semiconductor manufactured by a vapor-phase growth method using a semiconductor source gas typified by silane or germane or a sputtering method; a polycrystalline semiconductor formed by crystallizing the amorphous semiconductor with the use of light energy or thermal energy; a microcrystalline semiconductor; or the like. The semiconductor layer can be formed by a sputtering method, an LPCVD method, a plasma CVD method, or the like.

A typical example of an amorphous semiconductor is hydrogenated amorphous silicon, while a typical example of a crystalline semiconductor is polysilicon and the like. Polysilicon (polycrystalline silicon) includes what is called high-temperature polysilicon that contains, as its main component, polysilicon formed at a process temperature of 800° C. or higher, what is called low-temperature polysilicon that contains, as its main component, polysilicon formed at a process temperature of 600° C. or lower, and polysilicon formed by crystallizing amorphous silicon by using an element that promotes crystallization, or the like. Needless to say, as described above, a microcrystalline semiconductor or a semiconductor which includes a crystal phase in part of a semiconductor layer can also be used.

Alternatively, an oxide semiconductor may be used. Examples of an oxide semiconductor are an In—Sn—Ga—Zn—O-based oxide semiconductor, an In—Ga—Zn—O-based oxide semiconductor, an In—Sn—Zn—O-based oxide semiconductor, an In—Al—Zn—O-based oxide semiconductor, a Sn—Ga—Zn—O-based oxide semiconductor, an Al—Ga—Zn—O-based oxide semiconductor, a Sn—Al—Zn—O-based oxide semiconductor, an In—Zn—O-based oxide semiconductor, a Sn—Zn—O-based oxide semiconductor, an Al—Zn—O-based oxide semiconductor, a Zn—Mg—O-based oxide semiconductor, a Sn—Mg—O-based oxide semiconductor, an In—Mg—O-based oxide semiconductor, In—Ga—O-based oxide semiconductor, an In—O-based oxide semiconductor, a Sn—O-based oxide semiconductor, and a Zn—O-based oxide semiconductor. The above oxide semiconductor may contain SiO₂. Here, for example, the In—Ga—Zn—O-based oxide semiconductor means an oxide containing at least In, Ga, and Zn, and the composition thereof is not particularly limited. The In—Ga—Zn—O-based oxide semiconductor may contain an element other than In, Ga, and Zn.

For the oxide semiconductor layer, a thin film expressed by a chemical formula InMO₃(ZnO)ₘ (m>0) can be used. Here, M denotes one or more metal elements selected from Ga, Al, Mn, and Co. For example, M may be Ga, Ga and Al, Ga and Mn, Ga and Co, or the like.

As the oxide semiconductor layer, a CAAC-OS (c-axis aligned crystalline oxide semiconductor) film can be used, for example.

The CAAC-OS film is one of oxide semiconductor films having a plurality of c-axis aligned crystal parts.

In a process of forming the semiconductor layer and the wiring layer, an etching step is employed to process thin films into desired shapes. Dry etching or wet etching can be employed for the etching step.

As an etching apparatus used for the dry etching, an etching apparatus using a reactive ion etching method (an RIE method) or a dry etching apparatus using a high-density plasma source such as ECR (electron cyclotron resonance) or ICP (inductively coupled plasma) can be used. As a dry etching apparatus by which uniform electric discharge can be performed over a large area as compared to an ICP etching apparatus, there is an ECCP (enhanced capacitively coupled plasma) mode etching apparatus in which an upper electrode is grounded, a high-frequency power source at 13.56 MHz is connected to a lower electrode, and further a low-frequency power source at 3.2 MHz is connected to the lower electrode. This ECCP mode etching apparatus can be applied, for example, even when a substrate of the tenth generation with a side of larger than approximately 3 m is used.

In order to etch the films into desired shapes, the etching conditions (the amount of power applied to a coil-shaped electrode, the amount of power applied to an electrode on the substrate side, the temperature of the electrode on the substrate side, and the like) are adjusted as appropriate.

The etching conditions (such as an etchant, etching time, and temperature) are appropriately adjusted depending on the material so that the material can be etched to have a desired shape.

As a material of the wiring layers 405a and 405b serving as source and drain electrode layers, an element selected from Al, Cr, Ta, Ti, Mo, and W; an alloy containing any of the above elements as its component; an alloy film containing a combination of any of these elements; and the like can be given. Further, in the case where heat treatment is performed, the conductive film preferably has heat resistance against the heat treatment. For example, since use of Al alone brings disadvantages such as low heat resistance and a tendency to corrode, aluminum is used in combination with a conductive material having heat resistance. As the conductive material having heat resistance, which is combined with aluminum, it is possible to use an element selected from titanium (Ti), tantalum (Ta), tungsten (W), molybdenum (Mo), chromium (Cr), neodymium (Nd), and scandium (Sc); an alloy containing any of these elements as its component, an alloy containing a combination of any of these elements; or a nitride containing any of these elements as its component.

The gate insulating layer 402, the semiconductor layer 403, and the wiring layers 405a and 405b serving as source and drain electrode layers may be successively formed without being exposed to the air. When the gate insulating layer 402, the semiconductor layer 403, and the wiring layers 405a and 405b are formed successively without being exposed to the air, an interface between the layers can be formed without being contaminated with atmospheric components or impurity elements included in the air. Thus, variations in characteristics of transistors can be reduced.

Note that the semiconductor layer 403 is partly etched so as to have a groove (a depression portion).

As the insulating film 407 and the insulating film 409 which cover the transistor 420, an inorganic insulating film or an organic insulating film formed by a dry method or a wet method can be used. For example, it is possible to use a silicon nitride film, a silicon oxide film, a silicon oxynitride film, an aluminum oxide film, or a tantalum oxide film, which is formed by a CVD method, a sputtering method, or the like. It is also possible to use a low-dielectric constant material (a low-k material), a siloxane-based resin, PSG (phosphosilicate glass), BPSG (borophosphosilicate glass), or the like. A gallium oxide film may also be used as the insulating film 407. Alternatively, an organic material such as a polyimide, an acrylic resin, a benzocyclobutene-based resin, a polyamide, or an epoxy resin can be used.

Note that the siloxane-based resin is a resin including a Si—O—Si bond formed using a siloxane-based material as a starting material. The siloxane-based resin may include as a substituent an organic group (e.g., an alkyl group or an aryl group) or fluorine. In addition, the organic group may include fluorine. A siloxane-based resin is applied by a coating method and baked; thus, the insulating film 407 can be formed.

Alternatively, the insulating film 407 and the insulating film 409 may be formed by stacking a plurality of insulating films formed using any of these materials. For example, the insulating film 407 and the insulating film 409 may each have such a structure that an organic resin film is stacked over an inorganic insulating film.

Furthermore, with the use of a resist mask having regions with plural thicknesses (typically, two different thicknesses) which is formed using a multi-tone mask, the number of resist masks can be reduced, resulting in simplified process and lower cost.

The use of the liquid crystal composition exhibiting a blue phase and including the trifluorophenyl derivative and a chiral material contributes to higher contrast, so that a liquid crystal display device having a high level of visibility and high image quality can be provided.

The liquid crystal composition exhibiting a blue phase is capable of high-speed response. Thus, a high-performance liquid crystal display device can be achieved.

This embodiment can be implemented in an appropriate combination with any of the structures described in the other embodiments.

### Embodiment 4

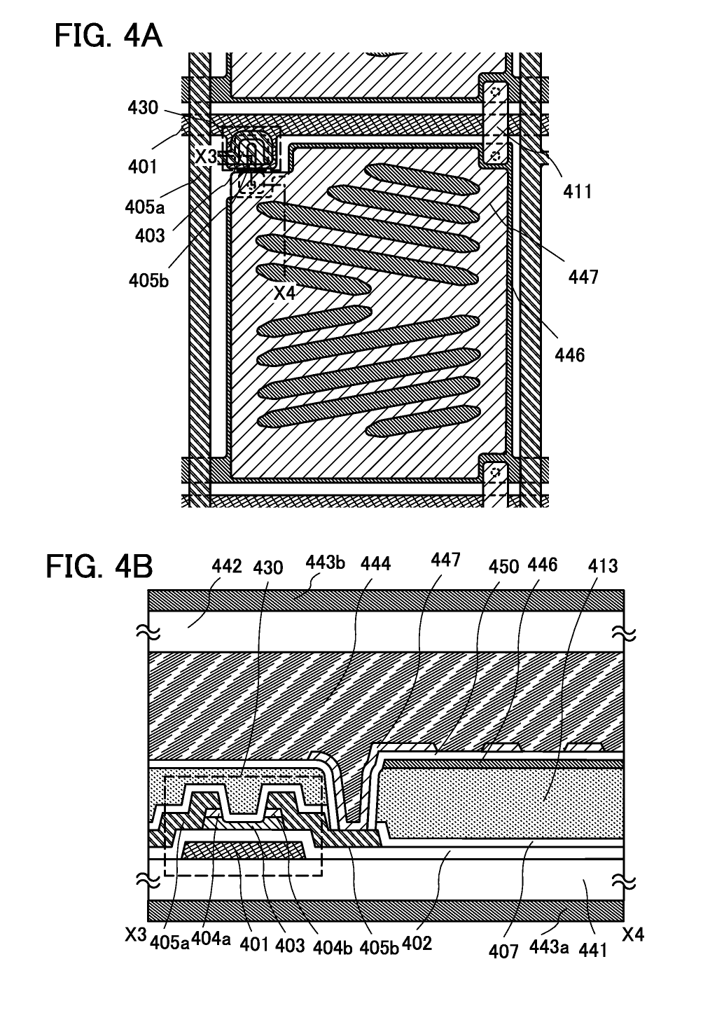

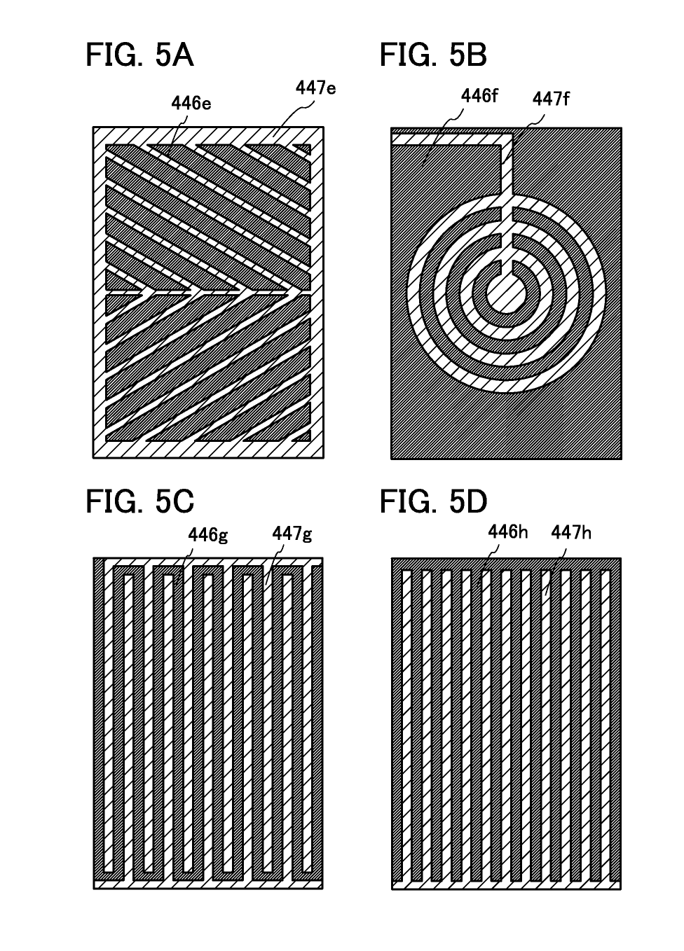

Another example of an active matrix liquid crystal display device to which the invention disclosed in this specification is applied will be described with reference to FIGS. 4A and 4B and FIGS. 5A to 5D.

FIG. 4A is a plan view of the liquid crystal display device and illustrates one pixel. FIG. 4B is a cross-sectional view along X3-X4 in FIG. 4A.

In FIG. 4A, a plurality of source wiring layers (including the wiring layer 405a) is arranged so as to be parallel to (extend in the longitudinal direction in the drawing) and apart from each other. A plurality of gate wiring layers (including the gate electrode layer 401) is arranged so as to be extended in a direction perpendicular to or substantially perpendicular to the source wiring layers (the horizontal direction in the drawing) and apart from each other. Common wiring layers (common electrode layers) are provided so as to be adjacent to the corresponding gate wiring layers and extended in a direction parallel to or substantially parallel to the gate wiring layers, that is, in a direction perpendicular to or substantially perpendicular to the source wiring layers (the horizontal direction in the drawing). A roughly rectangular space is surrounded by the source wiring layers, the common wiring layer (the common electrode layer), and the gate wiring layer. In this space, a pixel electrode layer and a common electrode layer of the liquid crystal display device are provided. A transistor 430 for driving the pixel electrode layer is provided at an upper left corner of the drawing. A plurality of pixel electrode layers and a plurality of transistors are arranged in matrix.

In the liquid crystal display device in FIGS. 4A and 4B, the first electrode layer 447 electrically connected to the transistor 430 serves as a pixel electrode layer, while the second electrode layer 446 electrically connected to the common wiring layer serves as a common electrode layer. As illustrated in FIGS. 4A and 4B, the second electrode layer 446 also serves as the common wiring layer in the pixel; thus, adjacent pixels are electrically connected to each other with a common electrode layer 411. Note that a capacitor is formed with the pixel electrode layer and the common electrode layer. Although the common electrode layer can operate in a floating state (an electrically isolated state), the potential of the common electrode layer may be set to a fixed potential, preferably to a potential around a common potential (an intermediate potential of an image signal which is transmitted as data) at such a level as not to generate flickers.

A method can be used in which the gray scale is controlled by generating an electric field parallel to or substantially parallel to a substrate (i.e., in the lateral direction) to move liquid crystal molecules in a plane parallel to the substrate. For such a method, an electrode structure used in an FFS mode illustrated in FIGS. 4A and 4B and FIGS. 5A to 5D can be employed.

In a lateral electric field mode such as an FFS mode, a first electrode layer (e.g., a pixel electrode layer with which a voltage is controlled in each pixel) having an opening pattern is located below a liquid crystal composition, and further, a second electrode layer (e.g., a common electrode layer with which a common voltage is applied to all pixels) having a flat shape is located below the opening pattern. Therefore, the first electrode layer 447 and the second electrode layer 446, one of which is a pixel electrode layer and the other of which is a common electrode layer, are formed over the first substrate 441, and the pixel electrode layer and the common electrode layer are stacked with an insulating film (or an interlayer insulating film) interposed therebetween. One of the pixel electrode layer and the common electrode layer is formed below the other and has a flat shape, whereas the other is formed above the one and has various opening patterns including a bent portion or a branched comb-like portion. In order to generate an electric field substantially parallel to a substrate between the first electrode layer 447 and the second electrode layer 446, an arrangement is avoided in which they have the same shape and completely overlap with each other.

In this embodiment, an electrode layer having an opening pattern (slit) is used as the first electrode layer 447 which is a pixel electrode layer, and an electrode layer having a flat shape is used as the second electrode layer 446 which is a common electrode layer.

In order to generate an electric field between the first electrode layer 447 and the second electrode layer 446, the electrode layers are located such that the second electrode layer 446 having a flat shape and the opening pattern (slit) of the first electrode layer 447 overlap with each other.

As the liquid crystal composition 444, the liquid crystal composition exhibiting a blue phase and including the trifluorophenyl derivative shown in Embodiment 1 and a chiral material is used.

With a lateral electric field generated between the first electrode layer 447 and the second electrode layer 446, liquid crystal of the liquid crystal composition 444 is controlled. Hence, a wide viewing angle can be obtained.

FIGS. 5A to 5D illustrate examples of the first electrode layer 447 and the second electrode layer 446. As illustrated in FIGS. 5A to 5D, first electrode layers 447e to 447h and second electrode layers 446e to 446h are disposed so as to overlap with each other, and insulating films are formed between the first electrode layers 447e to 447h and the second electrode layers 446e to 446h, so that the first electrode layers 447e to 447h and the second electrode layers 446e to 446h are formed over different films.

As illustrated in top views in FIGS. 5A to 5D, the first electrode layers 447e to 447h are formed in various shapes over the second electrode layers 446e to 446h. In FIG. 5A, the first electrode layers 447e is formed in a V-like shape over the second electrode layer 446e; in FIG. 5B, the first electrode layer 447f is formed in a concentric circular shape over the second electrode layer 446f; in FIG. 5C, the first electrode layer 447g which is formed over the second electrode layer 446g is formed in a comb-like shape and the first electrode layer 447g and the second electrode layer 446g are engaged with each other; and in FIG. 5D, the first electrode layer 447h is formed in a comb-like shape over the second electrode layer 446h.

The transistor 430 is an inverted staggered thin film transistor in which the gate electrode layer 401, the gate insulating layer 402, the semiconductor layer 403, source and drain regions 404a and 404b, and the wiring layers 405a and 405b which function as a source electrode layer and a drain electrode layer are formed over the first substrate 441 which has an insulating surface. The first electrode layer 447 is formed in the same layer as the gate electrode layer 401 over the first substrate 441 and is an electrode layer having a flat shape in the pixel.

As in the transistor 430, the source and drain regions 404a and 404b may be provided between the semiconductor layer 403 and the wiring layers 405a and 405b which function as a source electrode layer and a drain electrode layer. The source and drain regions 404a and 404b may be formed using a semiconductor layer whose resistance is lower than that of the semiconductor layer 403, or the like.

The insulating film 407 which covers the transistor 430 and is in contact with the semiconductor layer 403 is provided. The interlayer film 413 is provided over the insulating film 407, the second electrode layer 446 in a flat shape is provided in a pixel over the interlayer film 413, and the first electrode layer 447 having an opening pattern is formed over the second electrode layer 446 with the insulating film 450 interposed therebetween. Thus, the first electrode layer 447 and the second electrode layer 446 are provided so as to overlap with each other with the insulating film 450 interposed therebetween.

Note that in this embodiment, with the use of light-transmitting electrode layers for the first electrode layer 447 and the second electrode layer 446, a transmissive liquid crystal display device can be obtained. Alternatively, with the use of a reflective electrode layer for the second electrode layer 446 in a flat shape, a reflective liquid crystal display device can be obtained.

The use of the liquid crystal composition exhibiting a blue phase and including the trifluorophenyl derivative and a chiral material contributes to higher contrast, so that a liquid crystal display device having a high level of visibility and high image quality can be provided.

The liquid crystal composition exhibiting a blue phase is capable of high-speed response. Thus, a high-performance liquid crystal display device can be achieved.

This embodiment can be implemented in an appropriate combination with any of the structures described in the other embodiments.

### Embodiment 5

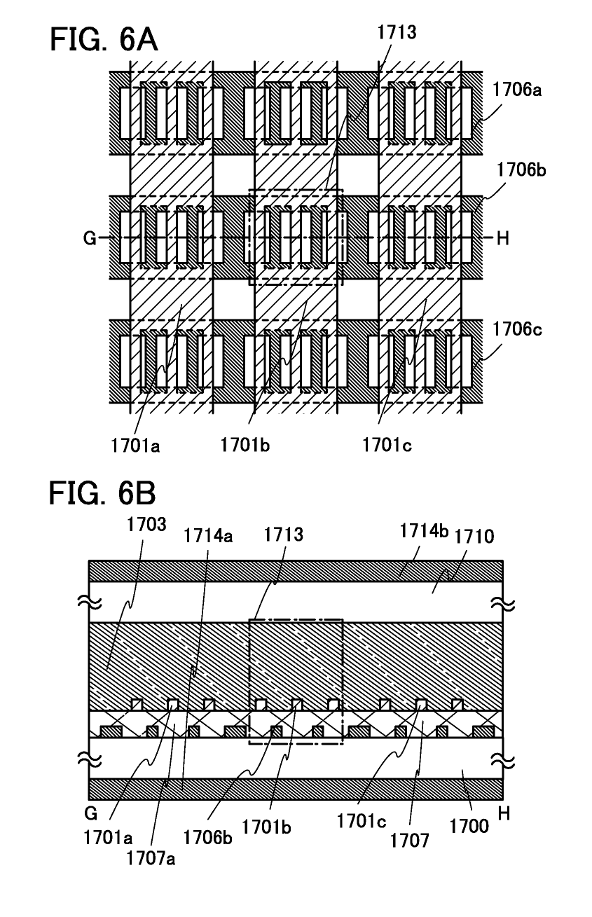

The invention disclosed in this specification can be applied to both a passive matrix liquid crystal display device and an active matrix liquid crystal display device. An example of a passive matrix liquid crystal display device will be described with reference to FIGS. 6A and 6B. FIG. 6A is a top view of a liquid crystal display device, and FIG. 6B is a cross-sectional view along G-H in FIG. 6A. In FIG. 6A, a liquid crystal composition 1703, a substrate 1710 which functions as a counter substrate, a polarizing plate 1714, and the like are omitted and not illustrated; however, they are provided as illustrated in FIG. 6B.

FIGS. 6A and 6B illustrate the liquid crystal display device in which a substrate 1700 that is provided with the polarizing plate 1714a and the substrate 1710 that is provided with the polarizing plate 1714b are positioned so as to face each other with the liquid crystal composition 1703 interposed therebetween. Common electrode layers 1706a, 1706b, and 1706c, an insulating film 1707, and pixel electrode layers 1701a, 1701b, and 1701c are provided between the substrate 1700 and the liquid crystal composition 1703.

The pixel electrode layers 1701a, 1701b, and 1701c and the common electrode layers 1706a, 1706b, and 1706c each have a shape with an opening pattern which includes a rectangular opening (slit) in a pixel region of a liquid crystal element 1713.

As the liquid crystal composition 1703, the liquid crystal composition exhibiting a blue phase and including trifluorophenyl derivative described in Embodiment 1 and a chiral material is used. The liquid crystal composition 1703 may contain an organic resin.

With a lateral electric field generated between the pixel electrode layers 1701a, 1701b, and 1701c and the common electrode layers 1706a, 1706b, and 1706c, liquid crystal of the liquid crystal composition 1703 is controlled. Hence, a wide viewing angle can be obtained.

In addition, a coloring layer which functions as a color filter may be provided, and the color filter may be provided on the inner side of the substrate 1700 or/and the substrate 1710 with respect to the liquid crystal composition 1703, between the substrate 1710 and the polarizing plate 1714b, or between the substrate 1700 and the polarizing plate 1714a.

When the liquid crystal display device performs full-color display, the color filter may be made of materials which exhibit red (R), green (G), and blue (B). When the liquid crystal display device performs single-color display, the coloring layer may be omitted or may be formed of a material which exhibits at least one color. Note that the color filter is not always provided in the case where light-emitting diodes (LEDs) of RGB, or the like are arranged in a backlight unit and a successive additive color mixing method (a field sequential method) in which color display is performed by time division is employed.

The pixel electrode layers 1701a, 1701b, and 1701c and the common electrode layers 1706a, 1706b and 1706c may be formed with the use of one or more of the following: indium tin oxide (ITO); a conductive material in which zinc oxide (ZnO) is mixed into indium oxide; a conductive material in which silicon oxide (SiO₂) is mixed into indium oxide; indium oxide containing tungsten oxide; indium zinc oxide containing tungsten oxide; indium oxide containing titanium oxide; and indium tin oxide containing titanium oxide; graphene; metals such as tungsten (W), molybdenum (Mo), zirconium (Zr), hafnium (Hf), vanadium (V), niobium (Nb), tantalum (Ta), chromium (Cr), cobalt (Co), nickel (Ni), titanium (Ti), platinum (Pt), aluminum (Al), copper (Cu), and silver (Ag); alloys thereof; and metal nitrides thereof.

The use of the liquid crystal composition exhibiting a blue phase and including the trifluorophenyl derivative and a chiral material contributes to higher contrast, so that a liquid crystal display device having a high level of visibility and high image quality can be provided.

The liquid crystal composition exhibiting a blue phase is capable of high-speed response. Thus, a high-performance liquid crystal display device can be achieved.

This embodiment can be implemented in an appropriate combination with any of the structures described in the other embodiments.

### Embodiment 6

The liquid crystal display device illustrated in any of Embodiments 2 to 5 can be provided with a light-blocking layer (a black matrix). Note that components similar to those in Embodiments 2 to 5 can be formed using similar materials and similar manufacturing methods, and detailed description of the same portions and portions which have similar functions is omitted.

The light-blocking layer may be provided on the inner side of a pair of substrates firmly attached to each other with a liquid crystal composition interposed therebetween or may be provided on the outer side of the substrates (on the side opposite to the liquid crystal composition).

In the case where a light-blocking layer is provided on the inner side of a pair of substrates in a liquid crystal display device, the light-blocking layer can be formed on the side of an element substrate provided with a pixel electrode layer, or on the counter substrate side. The light-blocking layer can be additionally provided; alternatively, in the case of an active matrix liquid crystal display device in Embodiment 3 or 4, the light-blocking layer can be formed as an interlayer film provided on an element substrate. In the liquid crystal display device of Embodiment 4 illustrated in FIGS. 4A and 4B, for example, a light-blocking layer can be formed as part of the interlayer film 413.

The light-blocking layer is formed using a light-blocking material that reflects or absorbs light. For example, a black organic resin can be used, which can be formed by mixing a black resin of a pigment material, carbon black, titanium black, or the like into a resin material such as photosensitive or non-photosensitive polyimide. Alternatively, a light-blocking metal film can be used, which may be formed using chromium, molybdenum, nickel, titanium, cobalt, copper, tungsten, aluminum, or the like, for example.

There is no particular limitation on the method for forming the light-blocking layer, and a dry method such as an evaporation method, a sputtering method, or a CVD method or a wet method such as spin coating, dip coating, spray coating, a droplet discharging method (e.g., ink-jetting), a printing method (e.g., screen printing or offset printing), may be used depending on the material. As needed, an etching method (dry etching or wet etching) may be employed to form a desired pattern.

In the case where the light-blocking layer is formed as part of the interlayer film 413, it is preferably formed using a black organic resin.

In the case where the light-blocking layer is formed directly on the element substrate side as part of the interlayer film, the problem of misalignment between the light-blocking layer and a pixel region does not occur, whereby the formation region can be controlled more precisely even when a pixel has a minute pattern.

When the liquid crystal display device has a structure in which the light-blocking layer is formed over the element substrate, light emitted from the counter substrate side is not absorbed or blocked by the light-blocking composition in light irradiation for polymer stabilization treatment; thus, the entire liquid crystal composition can be uniformly irradiated with light. Thus, alignment disorder of liquid crystal due to nonuniform photopolymerization, display unevenness due to the alignment disorder, and the like can be prevented.

In the liquid crystal display device, the light-blocking layer can be provided in an area overlapping with a semiconductor layer of a transistor or a contact hole, or between pixels.

The light-blocking layer provided in this manner can block light entering the semiconductor layer of the transistor; consequently, electric characteristics of the transistor can be prevented from varying due to incident light and can be stabilized. Further, the light-blocking layer prevents light leakage to an adjacent pixel, and reduces display unevenness caused by light leakage or the like due to an alignment defect of liquid crystal which occurs easily over a contact hole. As a result, higher definition and higher reliability of the liquid crystal display device can be achieved.

This embodiment can be implemented in an appropriate combination with any of the structures described in the other embodiments.

### Embodiment 7

This embodiment shows an example of a liquid crystal display device performing color display. The liquid crystal display device described in any of Embodiments 2 to 6 can be provided with a color filter to perform color display. Note that components similar to those in Embodiments 2 to 6 can be formed using similar materials and similar manufacturing methods, and detailed description of the same portions and portions which have similar functions is omitted.

In the case where a liquid crystal display device performs full-color display, a color filter may be made of materials which exhibit red (R), green (G), and blue (B). In the case of mono-color display other than monochrome display, a color filter may be made of a material which exhibits at least one color.

Specifically, the liquid crystal display device is provided with a coloring layer serving as a color filter layer. The light-blocking layer may be provided on the inner side of a pair of substrates firmly attached to each other with a liquid crystal composition interposed therebetween or may be provided on the outer side of the substrates (on the side opposite to the liquid crystal composition).

First, description will be made of the case where a color filter layer is provided on the inner side of a pair of substrates in a liquid crystal display device. The color filter layer can be formed on the side of an element substrate provided with a pixel electrode layer, or on the counter substrate side. The color filter layer can be additionally provided; alternatively, in the case of an active matrix liquid crystal display device described in Embodiment 3 or 4, the color filter layer can be formed as an interlayer film provided on an element substrate. In the case of the liquid crystal display device of Embodiment 3 illustrated in FIGS. 2A and 2B, for example, a chromatic-color light-transmitting resin layer serving as a color filter layer can be used as the interlayer film 413.

In the case where the interlayer film is formed directly on the element substrate side as the color filter layer, the problem of misalignment between the color filter layer and a pixel region does not occur, whereby the formation region can be controlled more precisely even when a pixel has a minute pattern. In addition, the same insulating layer serves as the interlayer film and the color filter layer, which brings advantages of process simplification and cost reduction.

When the liquid crystal display device has a structure in which the color filter layer is formed over the element substrate, light emitted from the counter substrate side is not absorbed by the light-blocking composition in light irradiation for polymer stabilization treatment; thus, the entire liquid crystal composition can be uniformly irradiated with light. Thus, alignment disorder of liquid crystal due to nonuniform photopolymerization, display unevenness due to the alignment disorder, and the like can be prevented.

As the chromatic-color light-transmitting resin that can be used for the color filter layer, a photosensitive organic resin or a non-photosensitive organic resin can be used. Use of the photosensitive organic resin layer makes it possible to reduce the number of resist masks; thus, the process is simplified, which is preferable.

Chromatic colors are colors except achromatic colors such as black, gray, and white. The coloring layer is formed of a material which only transmits light colored with chromatic color in order to function as the color filter. As chromatic color, red, green, blue, or the like can be used. Alternatively, cyan, magenta, yellow, or the like may be used. “Transmitting only the chromatic color light” means that light transmitted through the coloring layer has a peak at the wavelength of the chromatic color light.

The thickness of the color filter layer may be controlled as appropriate in consideration of the relation between the concentration of the coloring material to be included and the transmittance of light.

In the case where the thickness of the chromatic-color light-transmitting resin layer varies depending on the color or in the case where there is unevenness due to a light-blocking layer or a transistor, an insulating layer which transmits light in the visible wavelength range (a so-called colorless and transparent insulating layer) may be stacked for planarization. The improved planarization allows favorable coverage with a pixel electrode layer or the like formed over the color filter layer, and a uniform gap (thickness) of a liquid crystal composition, whereby the visibility of the liquid crystal display device is increased and higher image quality can be achieved.

In the case where the color filter is provided on the outer side of a substrate, the color filter can be attached to the substrate with an adhesive layer or the like. In the case where the color filter is provided on the outer side of a counter substrate, polymer stabilization of a blue phase is performed by light irradiation, and then the color filter is provided on the outer side of the counter substrate.

As a light source, a backlight, a sidelight, or the like may be used. Light from the light source is emitted to the viewing side through the color filter, so that color display can be performed. As a light source, a cold cathode tube or a white light-emitting diode can be used. In addition, an optical member such as a reflection plate, a diffusion plate, a polarizing plate, or a retardation plate may be provided.

Thus, a color display function can be added to the liquid crystal display device with high contrast and low power consumption.

This embodiment can be implemented in an appropriate combination with any of the structures described in the other embodiments.

### Embodiment 8

A liquid crystal display device having a display function can be manufactured by using transistors in a pixel portion and further in a driver circuit. Further, part or the whole of the driver circuit can be formed over the same substrate as the pixel portion, whereby a system-on-panel can be obtained.

The liquid crystal display device includes a liquid crystal element (also referred to as a liquid crystal display element) as a display element.

A liquid crystal display module includes a panel in which a display element is sealed, and a component in which an IC or the like including a controller is mounted to the panel. One embodiment of the present invention also relates to an element substrate, which corresponds to one mode before the display element is completed in a manufacturing process of the liquid crystal display device, and the element substrate is provided with a means for supplying current to the display element in each of a plurality of pixels. Specifically, the element substrate may be in a state in which only a pixel electrode of the display element is provided, a state after formation of a conductive film to be a pixel electrode and before etching of the conductive film to form the pixel electrode, or any other states.

Note that a liquid crystal display device in this specification means an image display device or a light source (including a lighting device). Furthermore, a liquid crystal display device also refers to all the following display modules in some cases: a display module in which a connector, for example, a flexible printed circuit (FPC) or a tape carrier package (TCP) is attached to a liquid crystal display device, a display module in which a printed wiring board is provided at an end of a TCP, and a display module in which an integrated circuit (IC) is directly mounted on a liquid crystal display device by a chip on glass (COG) method.

The display module may include a touch sensor panel provided over the liquid crystal display device. Note that a panel for a touch sensor is not necessarily provided separately; the display module may include an in-cell or on-cell touch sensor panel in which, for example, an electrode for a touch sensor is provided on a counter substrate of the liquid crystal display device. Furthermore, the display module may include a backlight, an optical film (a polarizing plate, a retardation plate, or a luminance increasing film), and the like.

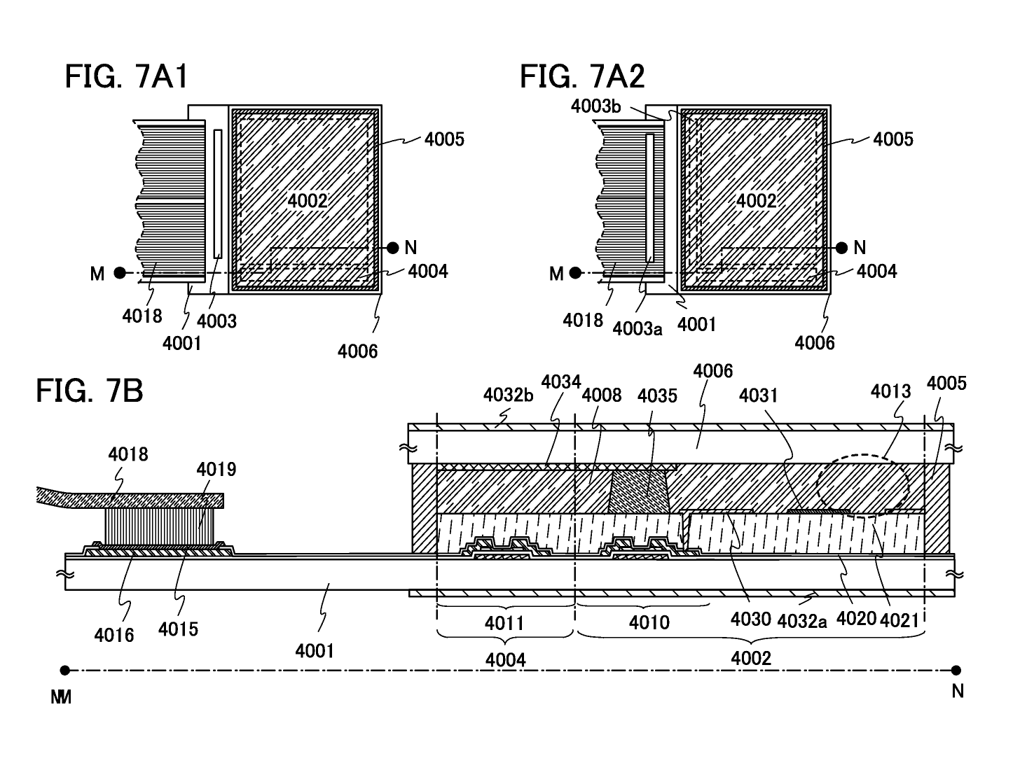

The appearance and a cross section of a liquid crystal display panel (a display module) which corresponds to a liquid crystal display device of one embodiment of the present invention will be described with reference to FIGS. 7A1, 7A2 and 7B. FIGS. 7A1 and 7A2 are top views of a panel in which transistors 4010 and 4011 and a liquid crystal element 4013 which are formed over a first substrate 4001 are sealed between the first substrate 4001 and a second substrate 4006 with a sealant 4005. FIG. 7B is a cross-sectional view taken along M-N of FIGS. 7A1 and 7A2.

The sealant 4005 is provided so as to surround a pixel portion 4002 and a scan line driver circuit 4004 which are provided over the first substrate 4001. The second substrate 4006 is provided over the pixel portion 4002 and the scan line driver circuit 4004. Thus, the pixel portion 4002 and the scan line driver circuit 4004 are sealed together with a liquid crystal composition 4008, by the first substrate 4001, the sealant 4005, and the second substrate 4006.

In FIG. 7A1, a signal line driver circuit 4003 that is formed using a single crystal semiconductor film or a polycrystalline semiconductor film over a substrate separately prepared is mounted in a region that is different from the region surrounded by the sealant 4005 over the first substrate 4001. FIG. 7A2 illustrates an example in which part of a signal line driver circuit is formed with the use of a transistor which is provided over the first substrate 4001. A signal line driver circuit 4003b is formed over the first substrate 4001 and a signal line driver circuit 4003a which is formed using a single crystal semiconductor film or a polycrystalline semiconductor film is mounted over a substrate separately prepared.

Note that there is no particular limitation on the connection method of a driver circuit which is separately formed, and a COG method, a wire bonding method, a TAB method, or the like can be used. FIG. 7A1 illustrates an example of mounting the signal line driver circuit 4003 by a COG method, and FIG. 7A2 illustrates an example of mounting the signal line driver circuit 4003 by a TAB method.

The pixel portion 4002 and the scan line driver circuit 4004 provided over the first substrate 4001 include a plurality of transistors. FIG. 7B illustrates the transistor 4010 included in the pixel portion 4002 and the transistor 4011 included in the scan line driver circuit 4004, as an example. An insulating layer 4020 and an interlayer film 4021 are provided over the transistors 4010 and 4011.

Any of the transistors shown in Embodiment 2 or 3 can be used as the transistors 4010 and 4011.

Further, a conductive layer may be provided over the interlayer film 4021 or the insulating layer 4020 so as to overlap with a channel formation region of a semiconductor layer of the transistor 4011 for the driver circuit. The conductive layer may have the same potential as or a potential different from that of a gate electrode layer of the transistor 4011 and can function as a second gate electrode layer. Further, the potential of the conductive layer may be GND, or the conductive layer may be in a floating state.

A pixel electrode layer 4030 and a common electrode layer 4031 are provided over the interlayer film 4021, and the pixel electrode layer 4030 is electrically connected to the transistor 4010. The liquid crystal element 4013 includes the pixel electrode layer 4030, the common electrode layer 4031, and the liquid crystal composition 4008. Note that a polarizing plate 4032a and a polarizing plate 4032b are provided on the outer sides of the first substrate 4001 and the second substrate 4006, respectively. In this embodiment, the pixel electrode layer 4030 and the common electrode layer 4031 have an opening pattern as illustrated in FIGS. 2A and 2B of Embodiment 2; however, one of the pixel electrode layer and the common electrode layer may be an electrode layer in a flat shape as in Embodiment 3. The structures of the pixel electrode layer and the common electrode layer, which are described in any of Embodiments 2 to 4, can be used.

As the liquid crystal composition 4008, the liquid crystal composition exhibiting a blue phase and including the trifluorophenyl derivative shown in Embodiment 1 and a chiral material is used. The liquid crystal composition provided as the liquid crystal composition 4008 may contain an organic resin.

With a lateral electric field generated between the pixel electrode layer 4030 and the common electrode layer 4031, liquid crystal of the liquid crystal composition 4008 is controlled. Hence, a wide viewing angle can be obtained.

As the first substrate 4001 and the second substrate 4006, glass, plastic, or the like having a light-transmitting property can be used. As plastic, a fiber-reinforced plastics (FRP) plate, a poly(vinyl fluoride) (PVF) film, a polyester film, or an acrylic resin film can be used. In addition, a sheet with a structure in which an aluminum foil is interposed between PVF films or polyester films can be used.

A columnar spacer denoted by reference numeral 4035 is obtained by selective etching of an insulating film and is provided in order to control the thickness (a cell gap) of the liquid crystal composition 4008. Alternatively, a spherical spacer may also be used. In the liquid crystal display device including the liquid crystal composition 4008, the cell gap which is the thickness of the liquid crystal composition is preferably greater than or equal to 1 μm and less than or equal to 20 μm. In this specification, the thickness of a cell gap refers to the length (film thickness) of a thickest part of a liquid crystal composition.

Although FIGS. 7A1, 7A2, and 7B illustrate examples of transmissive liquid crystal display devices, one embodiment of the present invention can also be applied to a transflective liquid crystal display device and a reflective liquid crystal display device.

In the example of the liquid crystal display device illustrated in FIGS. 7A1, 7A2, and 7B, the polarizing plates are provided on the outer sides of the first substrate 4001 and the second substrate 4006; however, the polarizing plate may be provided on the inner side of the substrates. The position of the polarizing plate may be determined as appropriate depending on the material of the polarizing plate and conditions of the manufacturing process. Furthermore, a light-blocking layer serving as a black matrix may be provided.

A color filter layer or a light-blocking layer may be formed as part of the interlayer film 4021. In FIGS. 7A1, 7A2, and 7B, a light-blocking layer 4034 is provided on the second substrate 4006 side so as to cover the transistors 4010 and 4011. With the provision of the light-blocking layer 4034, the contrast can be increased and the transistors can be more highly stabilized.

The transistor may be, but is not necessarily, covered with the insulating layer 4020 which functions as a protective film of the transistor.

Note that the protective film is provided to prevent entry of impurities such as organic substance, metal, or moisture existing in the air and is preferably a dense film. The protective film may be formed by a sputtering method to have a single-layer structure or a layered structure including any of a silicon oxide film, a silicon nitride film, a silicon oxynitride film, a silicon nitride oxide film, an aluminum oxide film, an aluminum nitride film, an aluminum oxynitride film, and an aluminum nitride oxide film.

Further, in the case of further forming a light-transmitting insulating layer as a planarizing insulating film, the light-transmitting insulating layer can be formed using an organic material having heat resistance, such as polyimide, acrylic, benzocyclobutene, polyamide, or epoxy. Other than such organic materials, it is possible to use a low-dielectric constant material (a low-k material), a siloxane-based resin, PSG (phosphosilicate glass), BPSG (borophosphosilicate glass), or the like.

There is no particular limitation on the method for forming the interlayer layer, and the following method can be employed depending on the material: spin coating, dip coating, spray coating, a droplet discharging method (e.g., ink-jetting), a printing method (e.g., screen printing or offset printing), roll coating, curtain coating, knife coating, or the like.

The pixel electrode layer 4030 and the common electrode layer 4031 can be formed using a light-transmitting conductive material such as indium oxide containing tungsten oxide, indium zinc oxide containing tungsten oxide, indium oxide containing titanium oxide, indium tin oxide containing titanium oxide, indium tin oxide, indium zinc oxide, indium tin oxide to which silicon oxide is added, or graphene.

The pixel electrode layer 4030 and the common electrode layer 4031 can be formed of one or more materials selected from metals such as tungsten (W), molybdenum (Mo), zirconium (Zr), hafnium (Hf), vanadium (V), niobium (Nb), tantalum (Ta), chromium (Cr), cobalt (Co), nickel (Ni), titanium (Ti), platinum (Pt), aluminum (Al), copper (Cu), and silver (Ag); alloys thereof and metal nitrides thereof.

The pixel electrode layer 4030 and the common electrode layer 4031 can be formed using a conductive composition including a conductive macromolecule (also referred to as a conductive polymer).

A variety of signals and potentials are supplied to the signal line driver circuit 4003 which is separately formed, the scan line driver circuit 4004, or the pixel portion 4002 from an FPC 4018.

Since the transistor is easily broken by static electricity or the like, a protection circuit for protecting the driver circuits is preferably provided over the same substrate as a gate line or a source line. The protection circuit is preferably formed using a nonlinear element.

In FIGS. 7A1, 7A2, and 7B, a connection terminal electrode 4015 is formed using the same conductive film as that of the pixel electrode layer 4030, and a terminal electrode 4016 is formed using the same conductive film as that of source and drain electrode layers of the transistors 4010 and 4011.

The connection terminal electrode 4015 is electrically connected to a terminal included in the FPC 4018 via an anisotropic conductive film 4019.

Although FIGS. 7A1, 7A2, and 7B illustrate an example in which the signal line driver circuit 4003 is formed separately and mounted on the first substrate 4001, one embodiment of the present invention is not limited to this structure. The scan line driver circuit may be separately formed and then mounted, or only part of the signal line driver circuit or part of the scan line driver circuit may be separately formed and then mounted.

The use of the liquid crystal composition exhibiting a blue phase and including the trifluorophenyl derivative and a chiral material contributes to higher contrast, so that a liquid crystal display device having a high level of visibility and high image quality can be provided.

The liquid crystal composition exhibiting a blue phase is capable of high-speed response. Thus, a high-performance liquid crystal display device can be achieved.

This embodiment can be implemented in an appropriate combination with any of the structures described in the other embodiments.

### Embodiment 9

A liquid crystal display device disclosed in this specification can be used for a variety of electronic appliances (including game machines). Examples of such electronic appliances include a television set (also referred to as a television or a television receiver), a monitor of a computer or the like, a camera such as a digital camera or a digital video camera, a digital photo frame, a mobile phone handset (also referred to as a mobile phone or a mobile phone device), a portable game machine, a personal digital assistant, an audio reproducing device, a large game machine such as a pinball machine, and the like.

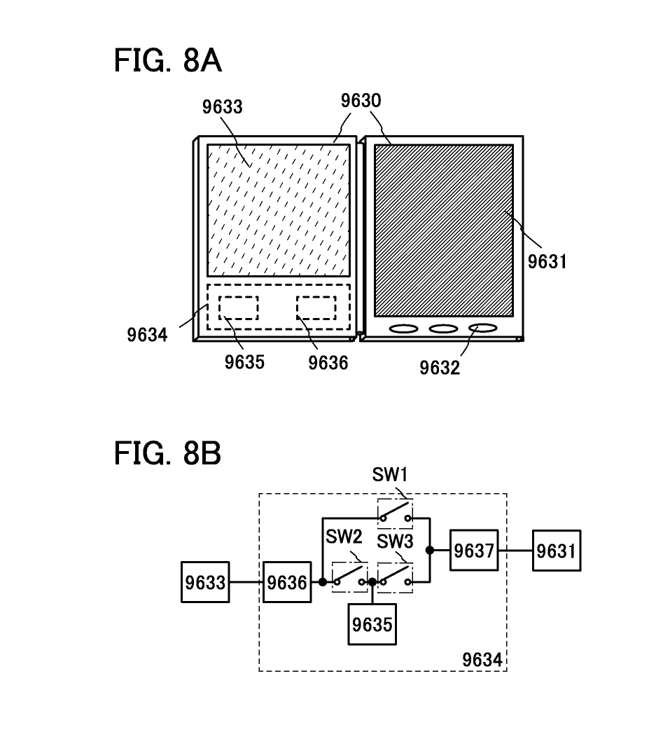

FIG. 8A illustrates an electronic book reader (also referred to as an e-book reader) which can include housings 9630, a display portion 9631, operation keys 9632, a solar cell 9633, and a charge and discharge control circuit 9634. The electronic book reader illustrated in FIG. 8A has a function of displaying various kinds of data (e.g., a still image, a moving image, and a text image) on the display portion, a function of displaying a calendar, a date, the time, or the like on the display portion, a function of operating or editing the data displayed on the display portion, a function of controlling processing by various kinds of software (programs), and the like. Note that in FIG. 8A, the charge and discharge control circuit 9634 has a battery 9635 and a DCDC converter (hereinafter, abbreviated as a converter) 9636. When the liquid crystal display device described in any of Embodiments 1 to 7 is used for the display portion 9631, the electronic book reader can have high contrast, a high level of visibility, and low power consumption.

In the case where a transflective liquid crystal display device or a reflective liquid crystal display device is used as the display portion 9631, use under a relatively bright condition is assumed; therefore, the structure illustrated in FIG. 8A is preferable because power generation by the solar cell 9633 and charge with the battery 9635 can be effectively performed. Since the solar cell 9633 can be provided in a space (a surface or a rear surface) of the housings 9630 as appropriate, the battery 9635 can be efficiently charged, which is preferable. When a lithium ion battery is used as the battery 9635, there is an advantage of downsizing or the like.

The structure and the operation of the charge and discharge control circuit 9634 illustrated in FIG. 8A will be described with reference to a block diagram in FIG. 8B. The solar cell 9633, the battery 9635, the converter 9636, a converter 9637, switches SW1 to SW3, and the display portion 9631 are shown in FIG. 8B, and the battery 9635, the converter 9636, the converter 9637, and the switches SW1 to SW3 are included in the charge and discharge control circuit 9634.

First, an example of operation in the case where power is generated by the solar cell 9633 using external light is described. The voltage of power generated by the solar cell is raised or lowered by the converter 9636 to a voltage for charging the battery 9635. Then, when the power from the solar cell 9633 is used for the operation of the display portion 9631, the switch SW1 is turned on and the voltage of the power is raised or lowered by the converter 9637 to a voltage needed for the display portion 9631. In addition, when display on the display portion 9631 is not performed, for example, the switch SW1 is turned off and the switch SW2 is turned on so that the battery 9635 is charged.

Next, operation in the case where power is not generated by the solar cell 9633 using external light is described. The voltage of power stored in the battery 9635 is raised or lowered by the converter 9637 by turning on the switch SW3. Then, power from the battery 9635 is used for the operation of the display portion 9631.

Note that although the solar cell 9633 is described as an example of a means for charge, the battery 9635 may be charged with another means. In addition, a combination of the solar cell 9633 and another means for charge may be used.

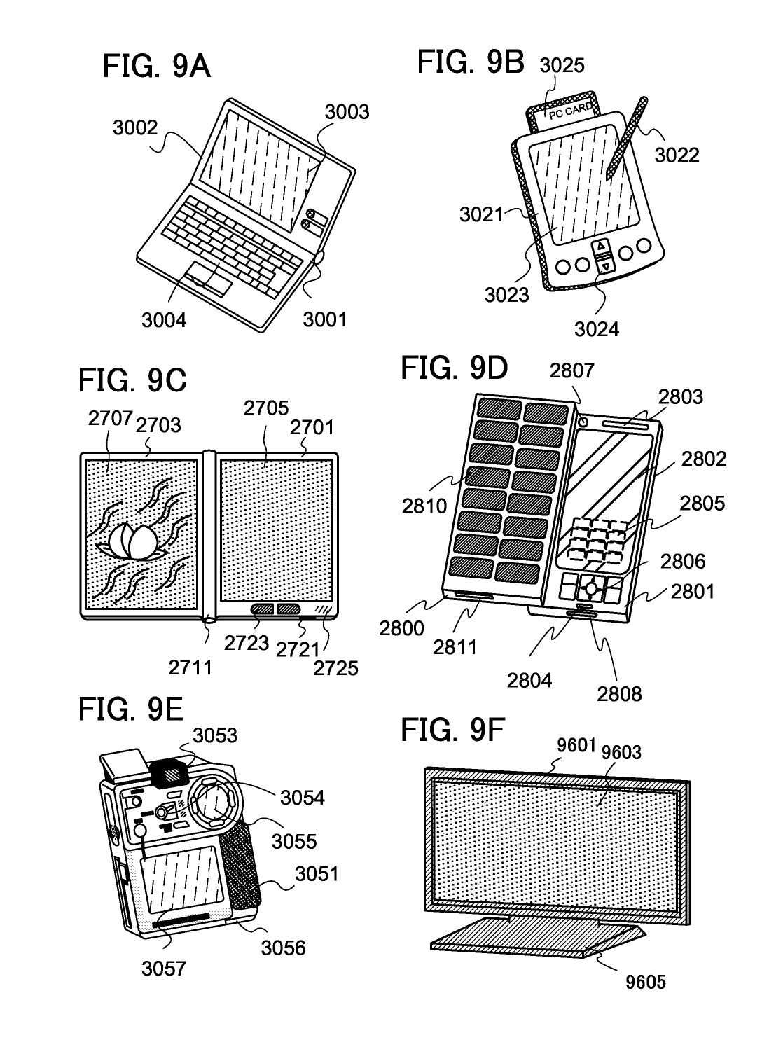

FIG. 9A illustrates a laptop personal computer, which includes a main body 3001, a housing 3002, a display portion 3003, a keyboard 3004, and the like. The liquid crystal display device described in any of Embodiments 1 to 7 is used for the display portion 3003, whereby the laptop personal computer can have high contrast, a high level of visibility, and high reliability.

FIG. 9B illustrates a personal digital assistant (PDA), which includes a main body 3021 provided with a display portion 3023, an external interface 3025, operation buttons 3024, and the like. A stylus 3022 is included as an accessory for operation. The liquid crystal display device described in any of Embodiments 1 to 7 is used for the display portion 3023, whereby the personal digital assistant can have high contrast, a high level of visibility, and high reliability.

FIG. 9C illustrates an e-book reader, which includes two housings, a housing 2701 and a housing 2703. The housing 2701 and the housing 2703 are combined with a hinge 2711 so that the e-book reader can be opened and closed with the hinge 2711 as an axis. With such a structure, the e-book reader can operate like a paper book.

A display portion 2705 and a display portion 2707 are incorporated in the housing 2701 and the housing 2703, respectively. The display portion 2705 and the display portion 2707 may display one image or different images. In the structure where different images are displayed in the above display portions, for example, the right display portion (the display portion 2705 in FIG. 9C) can display text and the left display portion (the display portion 2707 in FIG. 9C) can display images. The liquid crystal display device described in any of Embodiments 1 to 7 is used for the display portions 2705 and 2707, whereby the e-book reader can have high contrast, a high level of visibility, and high reliability.

FIG. 9C illustrates an example in which the housing 2701 is provided with an operation portion and the like. For example, the housing 2701 is provided with a power switch 2721, operation keys 2723, a speaker 2725, and the like. With the operation keys 2723, pages can be turned. Note that a keyboard, a pointing device, or the like may also be provided on the surface of the housing, on which the display portion is provided. An external connection terminal (an earphone terminal, a USB terminal, or the like), a recording medium insertion portion, and the like may be provided on the back surface or the side surface of the housing. Further, the e-book reader may have a function of an electronic dictionary.

The e-book reader may transmit and receive data wirelessly. Through wireless communication, desired book data or the like can be purchased and downloaded from an electronic book server.

FIG. 9D illustrates a mobile phone, which includes two housings, a housing 2800 and a housing 2801. The housing 2801 includes a display panel 2802, a speaker 2803, a microphone 2804, a pointing device 2806, a camera lens 2807, an external connection terminal 2808, and the like. The housing 2800 includes a solar cell 2810 for charging the mobile phone, an external memory slot 2811, and the like. Further, an antenna is incorporated in the housing 2801. The liquid crystal display device described in any of Embodiments 1 to 7 is used for the display panel 2802, whereby the mobile phone can have high contrast, a high level of visibility, and high reliability.

The display panel 2802 is provided with a touch panel. A plurality of operation keys 2805 which is displayed as images is illustrated by dashed lines in FIG. 9D. Note that a boosting circuit by which a voltage output from the solar cell 2810 is increased to be sufficiently high for each circuit is also included.

In the display panel 2802, the display direction can be appropriately changed depending on a usage pattern. The mobile phone is provided with the camera lens 2807 on the same surface as the display panel 2802, and thus it can be used as a video phone. The speaker 2803 and the microphone 2804 can be used for videophone calls, recording and playing sound, and the like as well as voice calls. Further, the housings 2800 and 2801 which are developed as illustrated in FIG. 9D can overlap with each other by sliding; thus, the size of the mobile phone can be decreased, which makes the mobile phone suitable for being carried.

The external connection terminal 2808 can be connected to an AC adapter and various types of cables such as a USB cable, and charging and data communication with a personal computer are possible. A large amount of data can be stored and can be moved by inserting a storage medium into the external memory slot 2811.

In addition to the above functions, an infrared communication function, a television reception function, or the like may be provided.

FIG. 9E illustrates a digital video camera, which includes a main body 3051, a display portion 3057, an eyepiece 3053, an operation switch 3054, a display portion 3055, a battery 3056, and the like. The liquid crystal display device described in any of Embodiments 1 to 7 is used for the display portion 3057 and the display portion 3055, whereby the digital video camera can have high contrast, a high level of visibility, and high reliability.

FIG. 9F illustrates a television device, which includes a housing 9601, a display portion 9603, and the like. The display portion 9603 can display images. Here, the housing 9601 is supported by a stand 9605. The liquid crystal display device described in any of Embodiments 1 to 7 is used for the display portion 9603, whereby the television device can have high contrast, a high level of visibility, and high reliability.

The television device can be operated with an operation switch of the housing 9601 or a separate remote controller. The remote controller may be provided with a display portion for displaying data output from the remote controller.

Note that the television device is provided with a receiver, a modem, and the like. With the use of the receiver, general television broadcasting can be received. Furthermore, when the television device is connected to a communication network by wired or wireless connection via the modem, one-way (from a transmitter to a receiver) or two-way (between a transmitter and a receiver, between receivers, or the like) data communication can be performed.

This embodiment can be implemented in an appropriate combination with any of the structures described in the other embodiments.

### Example 1

In this example, an example of synthesizing 3,4,5-trifluorophenyl 4-n-pentylbenzoate (abbreviation: PEP-5FFF) represented by Structural Formula (103) in Embodiment 1 will be described.

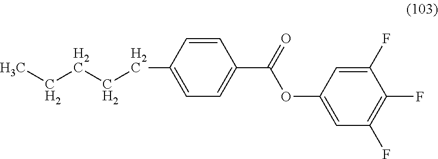 [SMILES: CCCCCc1ccc(C(=O)Oc2cc(F)c(F)c(F)c2)cc1]

### Step 1: Method of synthesizing 3,4,5-trifluorophenyl 4-n-pentylbenzoate (abbreviation: PEP-5FFF)

Into a 50-mL recovery flask were put 1.8 g (9.3 mmol) of 4-amyl benzoic acid, 1.4 g (9.3 mmol) of 3,4,5-trifluorophenol, 0.17 g (1.4 mmol) of N,N-dimethyl-N-(4-pyridinyl)amine, and 9.3 mL of dichloromethane, and the mixture was stirred. To this mixture was added 2.0 g (10 mmol) of 1-ethyl-3-(3-dimethylaminopropyl)carbodiimide hydrochloride (EDC), and the mixture was stirred under air at room temperature for 24 hours. To the resulting mixture was added water and an aqueous layer of this mixture was subjected to extraction with dichloromethane. The extracted solution and the organic layer were combined, and the mixture was washed with a saturated aqueous solution of sodium hydrogen carbonate and saturated saline and then dried with magnesium sulfate.

This mixture was gravity filtered, and the obtained filtrate was concentrated to give a yellow oily substance. This oily substance was purified by silica gel column chromatography (developing solvent: toluene). The obtained fraction was concentrated to give a light yellow solid. This solid was purified by high performance liquid column chromatography (HPLC) (developing solvent: chloroform). The obtained fraction was concentrated to give 0.78 g of a colorless oily substance of 3,4,5-trifluorophenyl 4-n-pentylbenzoate (abbreviation: PEP-5FFF) in a yield of 26%. The reaction scheme (E1-1) of Step 1 is shown below.

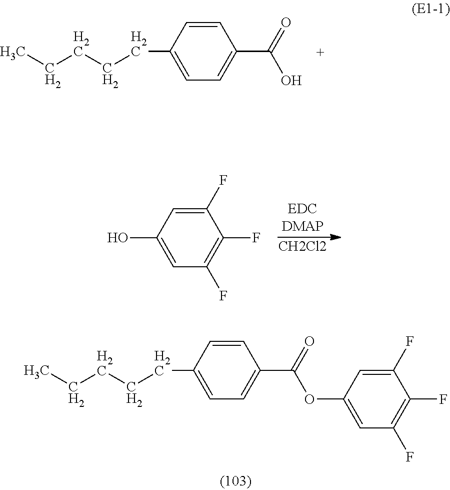

This compound was identified as 3,4,5-trifluorophenyl 4-n-pentylbenzoate (abbreviation: PEP-5FFF), which was the substance to be produced, by nuclear magnetic resonance (NMR) spectroscopy.

¹H NMR data of the obtained substance (PEP-5FFF) is as follows. ¹H NMR (CDCl₃, 300 MHz): δ (ppm)=0.90 (t, 3H), 1.27-1.42 (m, 4H), 1.61-1.71 (m, 2H), 2.73 (t, 2H), 6.88-6.98 (m, 2H), 7.32 (d, 2H), 8.06 (d, 2H).

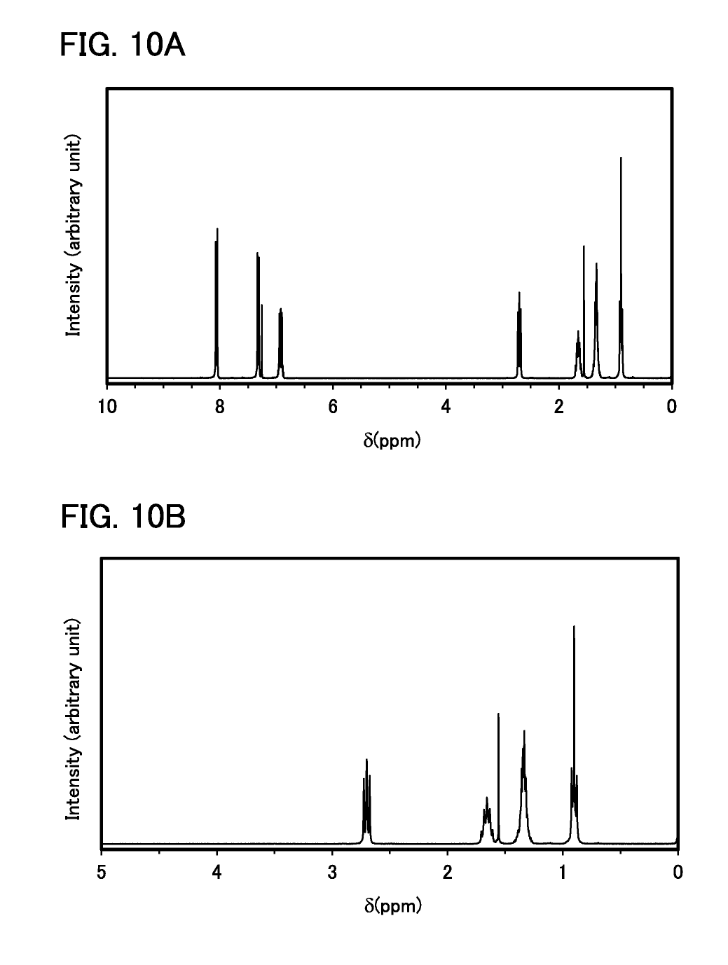

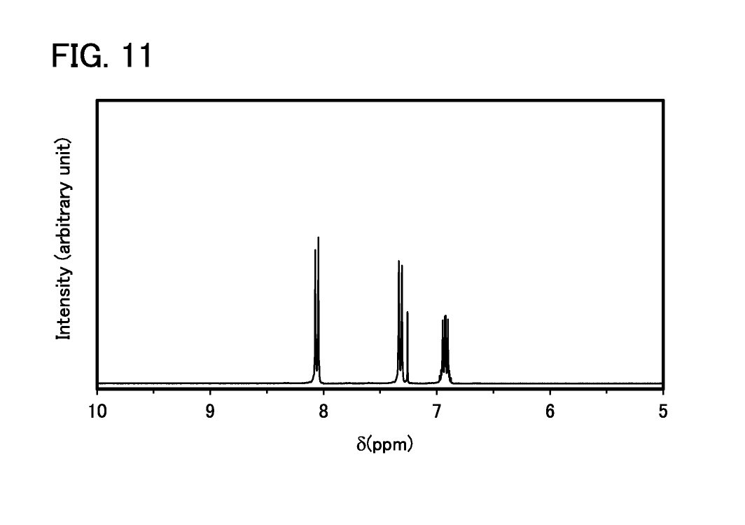

The ¹H NMR chart is shown in each of FIGS. 10A and 10B and FIG. 11. Note that FIG. 10B is an enlarged chart showing a range of 0 ppm to 5 ppm of FIG. 10A, and FIG. 11 is an enlarged chart showing a range of 5 ppm to 10 ppm of FIG. 10A. These results indicate that the target PEP-5FFF was obtained.

### Example 2

In this example, an example of a method of synthesizing 3,4,5-trifluorophenyl 4-[4-(n-heptyl)phenyl]benzoate (abbreviation: PPEP-7FFF) represented by Structural Formula (117) in Embodiment 1 will be described.

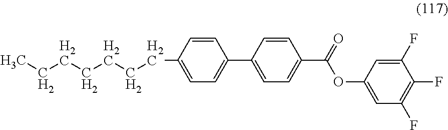 [SMILES: CCCCCCCc1ccc(-c2ccc(C(=O)Oc3cc(F)c(F)c(F)c3)cc2)cc1]

### Step 1: Method of synthesizing of 3,4,5-trifluorophenyl 4-[4-(n-heptyl)phenyl]benzoate (abbreviation: PPEP-7FFF)

Into a 50-mL recovery flask were put 2.1 g (9.3 mmol) of 4-[4-(n-heptyl)phenyl]benzoic acid, 1.0 g (7.0 mmol) of 3,4,5-trifluorophenol, 0.13 g (1.1 mmol) of N,N-dimethyl-N-(4-pyridinyl)amine, and 7.0 mL of dichloromethane, and the mixture was stirred. To this mixture was added 1.5 g (7.7 mmol) of 1-ethyl-3-(3-dimethylaminopropyl)carbodiimide hydrochloride (EDC), and stirring was performed in the atmosphere at room temperature for 24 hours. Water was added to the obtained mixture, and an aqueous layer of this mixture was subjected to extraction with dichloromethane. The extracted solution and an organic layer were combined, and the mixture was washed with a saturated aqueous solution of sodium hydrogen carbonate and saturated saline and then dried with magnesium sulfate.

This mixture was separated by gravity filtration, and the filtrate was concentrated to give a white solid. The obtained solid was purified by silica gel column chromatography (developing solvent: toluene). The obtained fraction was concentrated to give a light yellow solid. This solid was purified by high performance liquid column chromatography (HPLC) (developing solvent: chloroform). The obtained fraction was concentrated to give 2.1 g of the target white solid of 3,4,5-trifluorophenyl 4-[4-(n-heptyl)phenyl]benzoate in a yield of 70%. The reaction scheme (E2-1) of Step 1 is shown below.

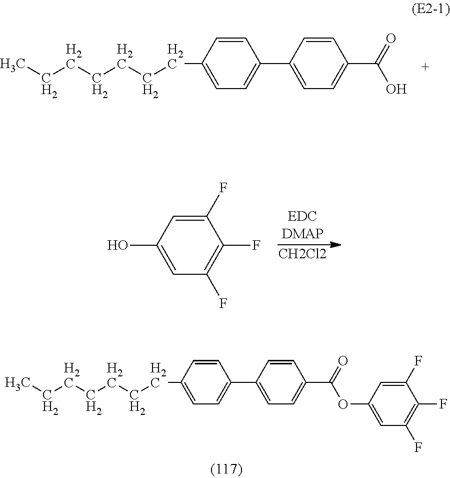

This compound was identified as 3,4,5-trifluorophenyl 4-[4-(n-heptyl)phenyl]benzoate (abbreviation: PPEP-7FFF), which was the target substance, by nuclear magnetic resonance (NMR) spectroscopy.

¹H NMR data of the obtained substance (PPEP-7FFF) is as follows. ¹H NMR (CDCl₃, 300 MHz): δ (ppm)=0.89 (t, 3H), 1.29-1.53 (m, 8H), 1.63-1.68 (m, 2H), 2.67 (t, 2H), 6.91-6.99 (m, 2H), 7.30 (d, 2H), 7.58 (d, 2H), 7.73 (d, 2H), 8.20 (d, 2H).

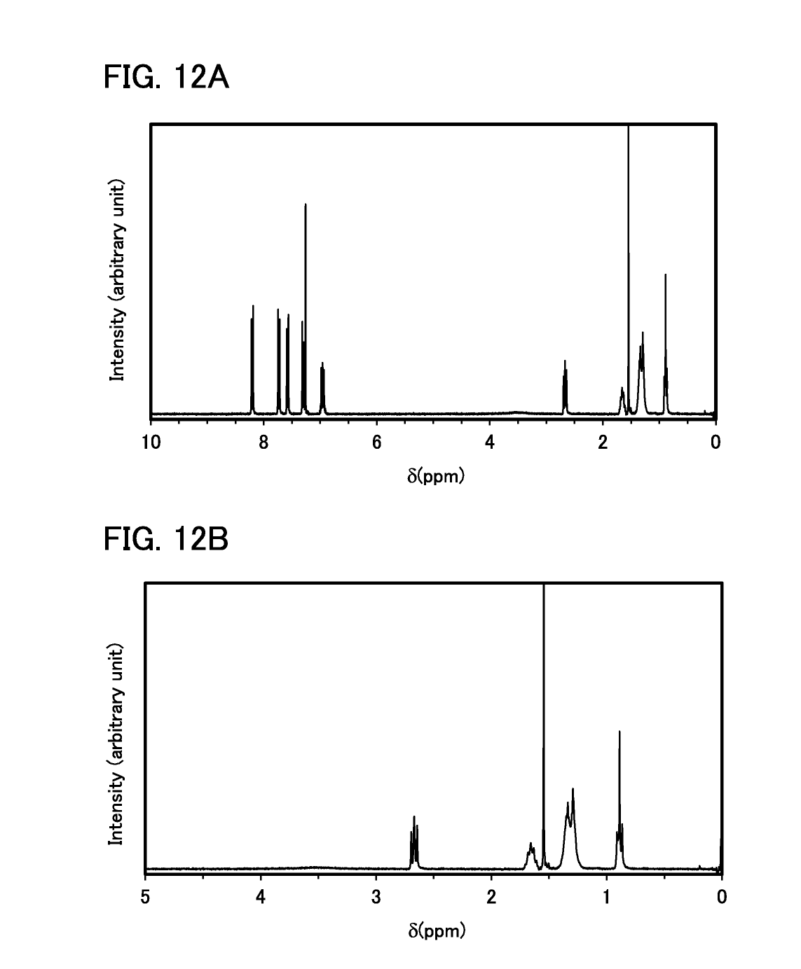

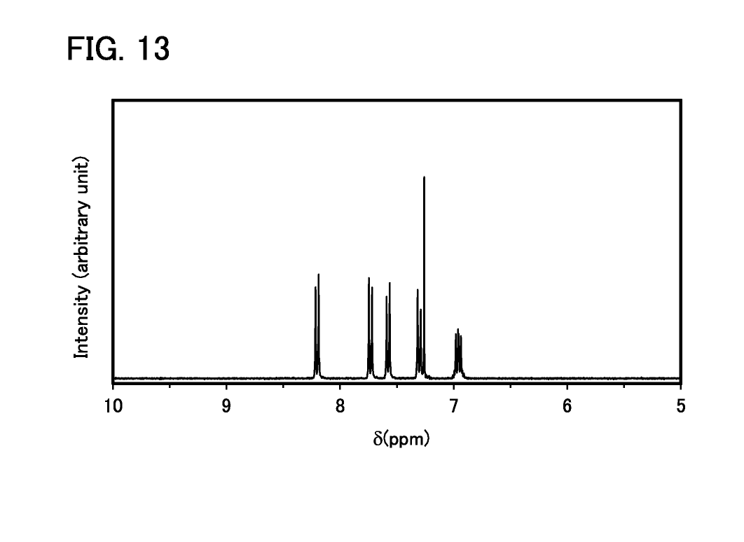

The ¹H NMR chart is shown in each of FIGS. 12A and 12B and FIG. 13. Note that FIG. 12B is an enlarged chart showing a range of 0 ppm to 5 ppm of FIG. 12A, and FIG. 13 is an enlarged chart showing a range of 5 ppm to 10 ppm of FIG. 12A. These results indicate that the target PPEP-7FFF was obtained.

### Example 3

In this example, an example of a method of synthesizing 3,4,5-trifluorophenyl 4-[4-(n-nonyl)phenyl]benzoate (abbreviation: PPEP-9FFF) represented by Structural Formula (119) in Embodiment 1 will be described.

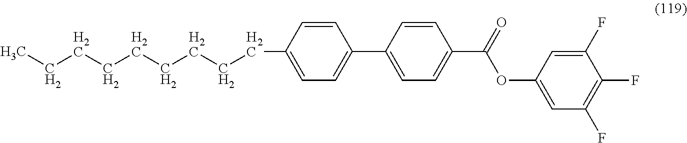 [SMILES: CCCCCCCCCc1ccc(-c2ccc(C(=O)Oc3cc(F)c(F)c(F)c3)cc2)cc1]

### Step 1: Method of synthesizing 3,4,5-trifluorophenyl 4-bromobenzoate

Into a 50-mL recovery flask were put 4.9 g (24 mmol) of 4-bromobenzoic acid, 3.6 g (24 mmol) of 3,4,5-trifluorophenol, 0.44 g (3.6 mmol) of N,N-dimethyl-N-(4-pyridinyl)amine, and 24 mL of dichloromethane, and the mixture was stirred. To this mixture was added 5.1 g (27 mmol) of 1-ethyl-3-(3-dimethylaminopropyl)carbodiimide hydrochloride (EDC), and stirring was performed in the atmosphere at room temperature for 5 hours. Water was added to the obtained mixture, and an aqueous layer of this mixture was subjected to extraction with dichloromethane. The extracted solution and an organic layer were combined, and the mixture was washed with a saturated aqueous solution of sodium hydrogen carbonate and saturated saline and then dried with magnesium sulfate.

This mixture was separated by gravity filtration, and the filtrate was concentrated to give a white solid. The obtained solid was purified by silica gel column chromatography (developing solvent: toluene). The obtained fraction was concentrated and dried in a vacuum to give a white solid. This solid was purified by high performance liquid column chromatography (HPLC) (developing solvent: chloroform). The obtained fraction was concentrated to give 7.5 g of the target white solid of 3,4,5-trifluorophenyl 4-bromobenzoate in a yield of 94%. The reaction scheme (E3-1) of Step 1 is shown below.

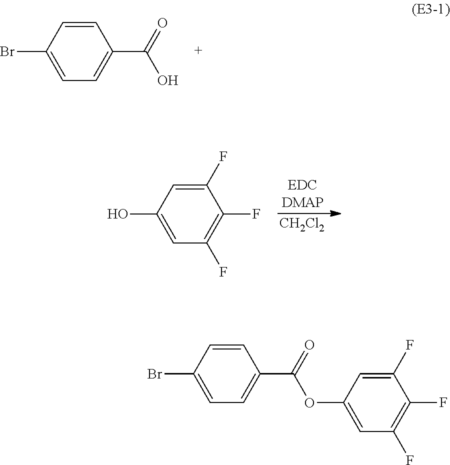

### Step 2: Method of synthesizing 3,4,5-trifluorophenyl 4-[4-(n-nonyl)phenyl]benzoate (abbreviation: PPEP-9FFF)

Into a flask were put 2.5 g (10 mmol) of 4-(n-nonyl)phenylboronic acid, 3.3 g (10 mmol) of 3,4,5-trifluorophenyl 4-bromobenzoate, 0.16 g (0.55 mmol) of tris(2-methylphenyl)phosphine, 10 mL of toluene, and 2.8 g of potassium carbonate. The mixture was degassed while being stirred under reduced pressure. After the degassing, the atmosphere in the flask was replaced with nitrogen. Into this mixture was added 23 mg (0.10 mmol) of palladium (II) acetate, and stirring was performed at 90° C. for 3.5 hours. To this mixture was added palladium (II) acetate and toluene, and the mixture was stirred at 90° C. for 11.5 hours. To the obtained mixture was added water, and an aqueous layer of this mixture was subjected to extraction with toluene. The extracted solution and an organic layer were combined, and the mixture was washed with saturated saline and then dried with magnesium sulfate.

This mixture was separated by gravity filtration and the filtrate was concentrated and suction-filtered through Celite (produced by Wako Pure Chemical Industries, Ltd., Catalog No. 531-16855), Florisil (produced by Wako Pure Chemical Industries, Ltd., Catalog No. 540-00135), and alumina. This mixture was purified by silica gel column chromatography (a mixed solvent of hexane:ethyl acetate=10:1). The resulting fraction was concentrated and dried in a vacuum to give a yellow solid.

This solid was purified by high performance liquid column chromatography (HPLC) (developing solvent: chloroform). The obtained fraction was concentrated to give 2.0 g of the target light yellow solid of 3,4,5-trifluorophenyl 4-[4-(n-nonyl)phenyl]benzoate (abbreviation: PPEP-9FFF) in a yield of 43%. The reaction scheme (E3-2) of Step 2 is shown below.

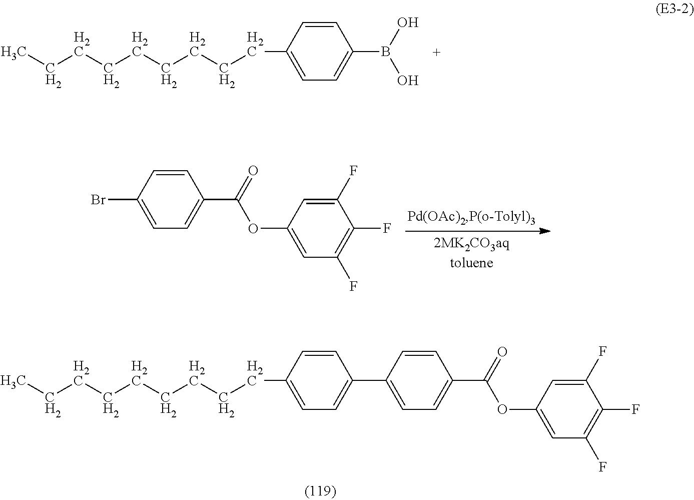 [SMILES: CCCCCCCCCc1ccc(B(O)O)cc1 ; O=C(Oc1cc(F)c(F)c(F)c1)c1ccc(Br)cc1 ; CCCCCCCCCc1ccc(-c2ccc(C(=O)Oc3cc(F)c(F)c(F)c3)cc2)cc1]

This compound was identified as 3,4,5-trifluorophenyl 4-[4-(n-nonyl)phenyl]benzoate (abbreviation: PPEP-9FFF), which was the target substance, by nuclear magnetic resonance (NMR) spectroscopy.

¹H NMR data of the obtained substance (PPEP-9FFF) is as follows. ¹H NMR (CDCl₃, 300 MHz): δ (ppm)=0.88 (t, 3H, 1.27-1.33 (m, 12H), 1.59-1.68 (m, 2H), 2.67 (t, 2H), 6.91-7.01 (m, 2H), 7.30 (d, 2H), 7.58 (d, 2H), 7.73 (d, 2H), 8.20 (d, 2H).

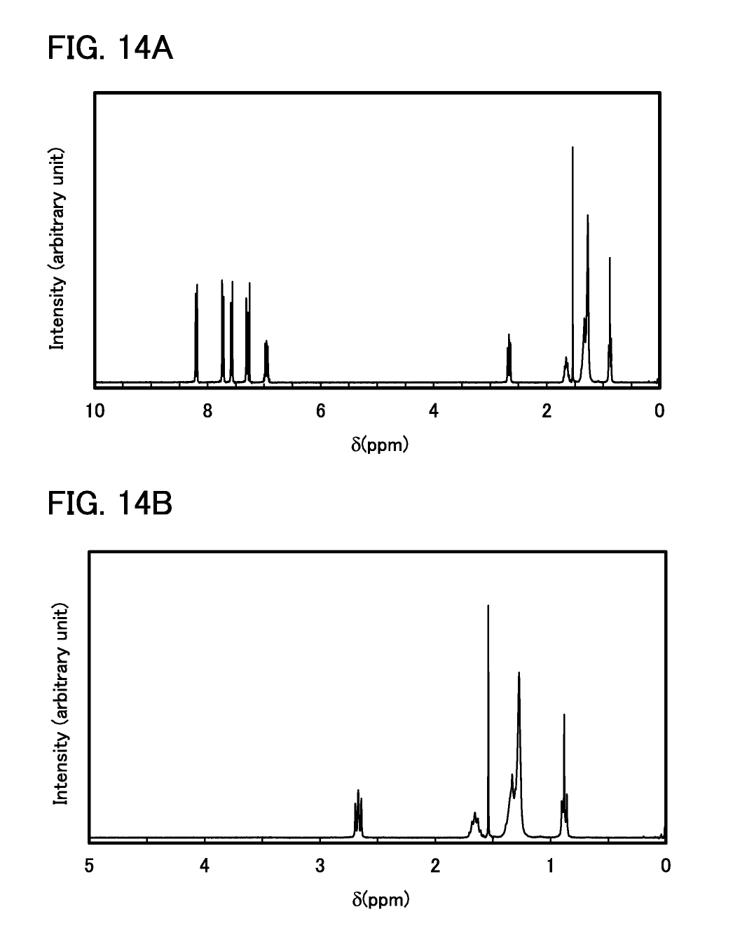

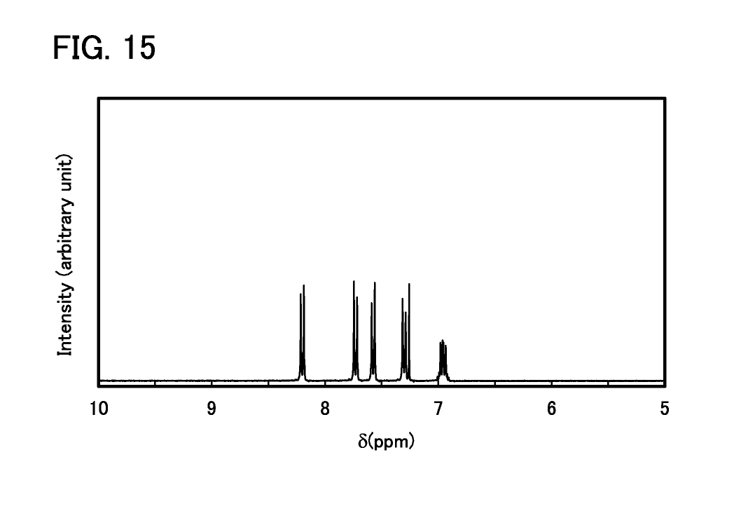

The ¹H NMR chart is shown in each of FIGS. 14A and 14B and FIG. 15. Note that FIG. 14B is an enlarged chart showing a range of 0 ppm to 5 ppm of FIG. 14A, and FIG. 15 is an enlarged chart showing a range of 5 ppm to 10 ppm of FIG. 14A. These results indicate that the target PPEP-9FFF was obtained.

### Example 4

In this example, the dielectric constant anisotropies of 3,4,5-trifluorophenyl 4-n-pentylbenzoate (abbreviation: PEP-5FFF) synthesized in Example 1, 3,4,5-trifluorophenyl 4-[4-(n-heptyl)phenyl]benzoate (abbreviation: PPEP-7FFF) synthesized in Example 2, and 3,4,5-trifluorophenyl 4-[4-(n-nonyl)phenyl]benzoate (abbreviation: PPEP-9FFF) synthesized in Example 3 were measured.

Table 1 shows ratios of liquid crystal materials for the liquid crystal compositions used in the liquid crystal elements manufactured in this example. In Table 1, the percentages (mixture ratios) are all indicated by weight. X represents PEP-5FFF, PPEP-7FFF, or PPEP-9FFF. Each of PEP-5FFF, PPEP-7FFF, and PPEP-9FFF was mixed with ZLI-4792 (produced by Merck), which is a mixed liquid crystal, at a ratio shown in Table 1, and each mixture was injected into a cell.

| | |
|---|---|
| | Mixture ratio |
| Liquid crystal | (wt %) |

| | | |
|---|---|---|
| ZLI-4792 | 95 | 90 |
| X | 5 | 10 |

Two types of cells were used for measuring the dielectric constant anisotropies: one is a vertical alignment cell with a cell thickness of 10 μm to which a vertical electric field can be applied; and the other is a horizontal alignment cell with a cell thickness of 10 μm to which a vertical electric field can be applied.

For fabricating the cells, EAGLE XG (produced by Corning Incorporated) was used as a substrate, and a 110-nm-thick layer of indium tin oxide containing silicon oxide (ITSO) was formed by a sputtering method as a pixel electrode layer. Then, patterning was performed on the pixel electrode layer such that the area of a pixel was 7 mm×7 mm. Furthermore, a horizontal alignment film (SE-7492, produced by Nissan Chemical Industries, Ltd.) or a vertical alignment film (SE-5661, produced by Nissan Chemical Industries, Ltd.) was formed to a thickness of approximately 70 nm on this substrate. Then, alignment treatment by a rubbing method was performed on only the substrate on which the horizontal alignment film was formed. Similarly, an alignment film was formed on a counter substrate. Then, spacers each having a diameter of 10 μm were dispersed on the substrate, and a thermosetting sealant (XN-651, produced by Mitsui Chemicals, Inc.) was disposed on the vicinity of pixels on the counter substrate. After that, the substrates were attached such that the alignment films faced to each other, and the sealant was baked at a pressure of 0.3 kgf/cm² at 160° C. for 5 hours; thus, the cells were completed. When the horizontal alignment cell is used, the substrates were attached such that the rubbing directions were not parallel.

The liquid crystal compositions shown in Table 1 were injected into the cells made in the above manner, and the capacitances of these samples were measured with EC-1 (produced by TOYO Corporation). A dielectric constant of horizontally aligned liquid crystals and a dielectric constant of vertically aligned liquid crystals were derived from the capacitances. After the dielectric constants of liquid crystal compositions each including PEP-5FFF, PPEP-7FFF, or PPEP-9FFF at 5 wt % or 10 wt % were obtained, the values were extrapolated to 100 wt %. From these values, the dielectric constant anisotropy of each liquid crystal (a difference in dielectric constant between the vertically aligned liquid crystal and the horizontally aligned liquid crystal) was obtained.

The dielectric constant anisotropies of PEP-5FFF, PPEP-7FFF, and PPEP-9FFF are 17.2, 21.1, and 24.2, respectively. These values are high enough to use in a nematic mode. The dielectric constant anisotropy of the host liquid crystal ZLI-4792 was 5.6, which was measured by the same method. The use of a liquid crystal with high dielectric constant anisotropy enables low voltage driving and improvement in response speed when voltage is applied; thus, by adding these liquid crystals, low voltage driving and improvement in response speed can be achieved in a nematic mode.

### Example 5

In this example, a liquid crystal composition including PPEP-7FFF (Sample 1), a liquid crystal composition including PPEP-9FFF (Sample 2), and a liquid crystal composition (Comparative sample 1) in which one embodiment of the present invention is not used were used to fabricate liquid crystal elements. The characteristics of these liquid crystal elements were evaluated.

Table 2, Table 3, and Table 4 show components of the liquid crystal compositions (Sample 1, Sample 2, and Comparative sample 1), respectively, included in the liquid crystal elements fabricated in this example. The percentages (mixture ratios) are all indicated by weight.

| Components | | | | Mixture ratio (wt %) | | | | |
|---|---|---|---|---|---|---|---|---|
| Sample B1 | Sample A1 | Liquid crystal | E-8 | 40.02 | 90.07 | 91.02 | 91.49 | 99.55 |
| | | | CPP-3FF | 29.98 | | | | |
| | | | PEP-5CNF | 30.00 | | | | |
| | | | PPEP-7FFF | | 9.93 | | | |
| | | Chiral material | ISO-(6OBA)₂ | | | 8.98 | | |
| | | Polymerizable monomer | RM-257-O6 | | | | 4.24 | |
| | | | DMeAc | | | | 4.27 | |
| | | Polymerization initiator | DMPAP | | | | | 0.45 |

| Components | | | | Mixture ratio (wt %) | | | | |
|---|---|---|---|---|---|---|---|---|
| Sample B2 | Sample A2 | Liquid crystal | E-8 | 40.02 | 89.95 | 90.73 | 91.28 | 99.67 |
| | | | CPP-3FF | 29.98 | | | | |
| | | | PEP-5CNF | 30.00 | | | | |
| | | | PPEP-9FFF | | 10.05 | | | |
| | | Chiral material | ISO-(6OBA)₂ | | | 9.27 | | |
| | | Polymerizable monomer | RM-257-O6 | | | | 4.44 | |
| | | | DMeAc | | | | 4.29 | |
| | | Polymerization initiator | DMPAP | | | | | 0.33 |

| Components | | | | Mixture ratio (wt %) | | | |
|---|---|---|---|---|---|---|---|
| Comparative | Comparative | Liquid crystal | E-8 | 40.02 | 91.08 | 91.6 | 99.48 |
| sample B1 | sample A1 | | CPP-3FF | 29.98 | | | |
| | | | PEP-5CNF | 30.00 | | | |
| | | Chiral material | ISO-(6OBA)₂ | | 8.92 | | |
| | | Polymerizable monomer | RM-257-O6 | | | 4.2 | |
| | | | DMeAc | | | 4.2 | |
| | | Polymerization initiator | DMPAP | | | | 0.52 |

Each of Sample A1, Sample A2, and Comparative sample A1 is a liquid crystal composition in which liquid crystals and a chiral material are mixed. As the chiral material, 1,4:3,6-dianhydro-2,5-bis[4-(n-hexyl-1-oxy)benzoic acid]sorbitol (abbreviation: ISO-(6OBA)2) (produced by Midori Kagaku Co., Ltd.) was used. As the liquid crystals, a mixed liquid crystal E-8 (produced by LCC Corporation, Ltd.), 4-(trans-4-n-propylcyclohexyl)-3′,4′-difluoro-1,1′-biphenyl (abbreviation: CPP-3FF) (produced by Daily Polymer Corporation), and 4-n-pentylbenzoic acid 4-cyano-3-fluorophenyl ester (abbreviation: PEP-5CNF) (produced by Daily Polymer Corporation) were used. In Sample A1 and Sample A2, PPEP-7FFF and PPEP-9FFF were respectively included. The amount of each of PPEP-7FFF and PPEP-9FFF was approximately 10 wt % of the total amount of the liquid crystals (E-8, CPP-3FF, and PEP-5CNF and PPEP-7FFF or PPEP-9FFF).

Sample B1, Sample B2, and Comparative sample B1 are liquid crystal compositions in which a polymerizable monomer and a polymerization initiator are added to Sample A1, Sample A2, and Comparative sample A1, respectively. As the polymerizable monomer, 1,4-bis-[4-(6-acryloyloxy-n-hexyl-1-oxy)benzoyloxy]-2-methylbenzene (abbreviation: RM257-O6) and dodecyl methacrylate (abbreviation: DMeAc) (produced by Tokyo Chemical Industry Co., Ltd.) were used. As the polymerization initiator, 2,2-dimethoxy-2-phenylacetophenone (abbreviation: DMPAP) (produced by Tokyo Chemical Industry Co., Ltd.) was used.

Structural Formulae of PPEP-7FFF, PPEP-9FFF, ISO-(6OBA)2, CPP-3FF, PEP-5CNF, RM257-O6, DMeAc, and DMPAP, which were used in this example, are shown below.

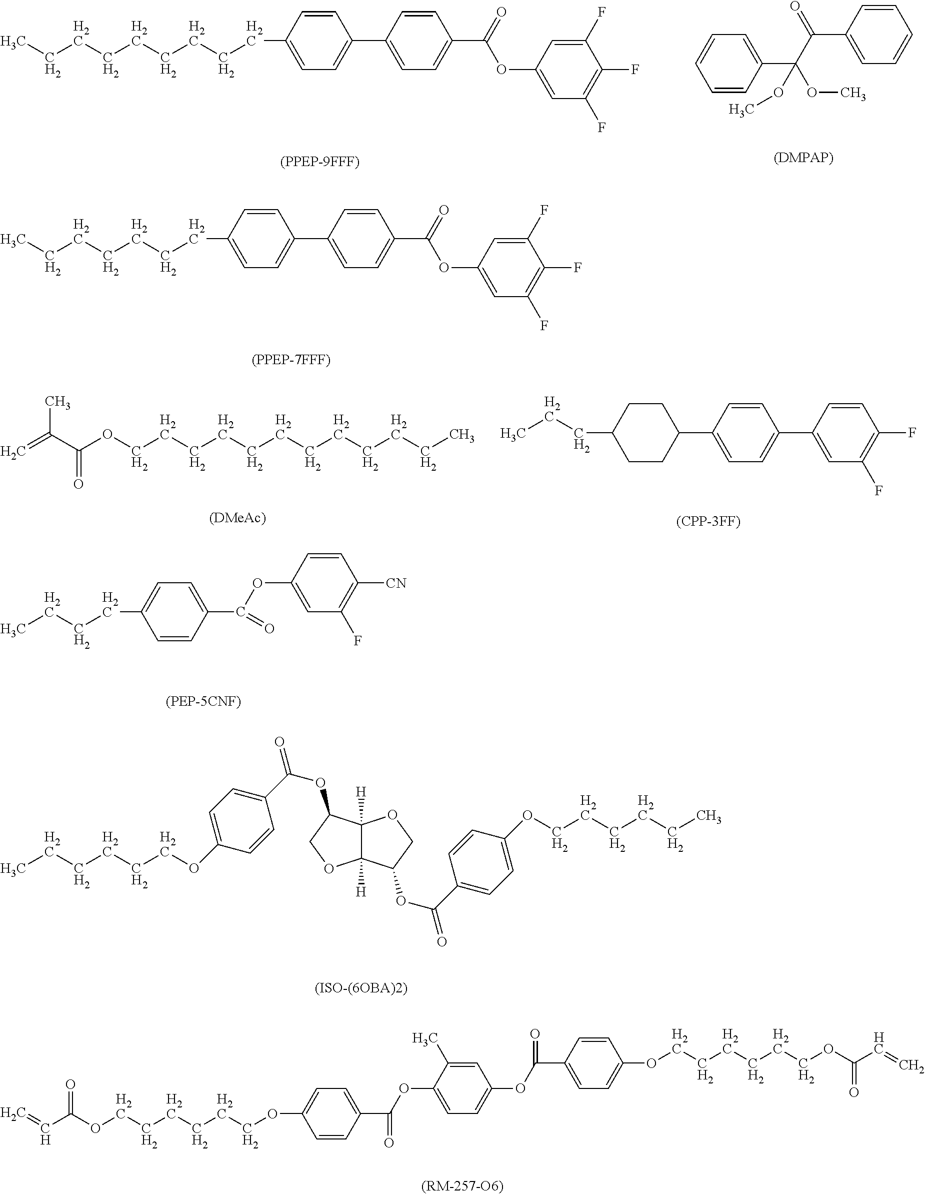

Each of the liquid crystal element including Sample A1, the liquid crystal element including Sample A2, and the liquid crystal element including Comparative sample A1 was fabricated in the following manner: a glass substrate over which a pixel electrode layer and a common electrode layer were formed in comb-like shapes and a glass substrate serving as a counter substrate were bonded to each other using sealant with a space (4 μm) provided therebetween; and the liquid crystal composition obtained by mixing materials in Table 2, Table 3, or Table 4 stirred in an isotropic phase at a ratio shown in Table 2, Table 3, or Table 4 was injected between the substrates by an injection method.

The pixel electrode layer and the common electrode layer were formed using indium tin oxide containing silicon oxide (ITSO) by a sputtering method. Note that the thickness was set to 110 nm, the width of the pixel electrode layer, the width of the common electrode layer, and the interval between the pixel electrode layer and the common electrode layer were each set to 2 μm. An ultraviolet light and heat curable sealant was used as the sealant. As curing treatment, ultraviolet (irradiance of 100 mW/cm²) irradiation was performed for 90 seconds, and then, heat treatment was performed at 120° C. for 1 hour.

First, the liquid crystal compositions in the liquid crystal elements each including Sample A1, Sample A2, or Comparative sample A1 were made to exhibit an isotropic phase. Then, the liquid crystal compositions were observed with the polarizing microscope while the temperature was decreased by 1.0° C. per minute with the temperature controller. In this manner, the temperature range where the liquid crystal compositions exhibit a blue phase was measured. The measurement conditions of the observation were as follows. In the polarizing microscope, a measurement mode was a reflective mode; polarizers were in crossed nicols; and the magnification was 50 times.

According to the results, the temperature range where a blue phase is exhibited is 45.5° C. to 43.1° C. in Sample A1, 44.7° C. to 42.9° C. in Sample A2, and 42.4° C. to 40.3° C. in Comparative sample A1. Thus, by including PPEP-7FFF or PPEP-9FFF, which is a liquid crystal material of one embodiment of the present invention, the upper limit of the temperature range can be high.

Next, reflectance spectra of the liquid crystal elements each including Sample A1, Sample A2, or Comparative sample A1 were measured. The measurement was performed using a polarizing microscope (MX-61L produced by Olympus Corporation), a temperature controller (HCS302-MK1000 produced by Instec, Inc.), and a microspectroscope (LVmicroUV/VIS produced by Lambda Vision Inc.).

The measurement conditions of the microspectroscope were as follows. A measurement mode was a reflective mode; polarizers were in crossed nicols; the measurement area was 12 μmφ; and the measurement wavelength was 250 nm to 800 nm. Note that the measurement was performed from the side of the glass substrate serving as the counter substrate, over which the pixel electrode layer and the common electrode layer were not formed, in order to avoid an influence of the electrode layers in measurement. Three to five arbitrary points were measured.

In the liquid crystal element including Sample A1 and exhibiting a blue phase, the average peak of a diffraction wavelength was 400 nm. In the case of Sample A2, the average peak was 391 nm. In the case of Comparative sample A1, the average peak was 412 nm. Consequently, with the use of PPEP-7FFF or PPEP-9FFF, which is a liquid crystal material of one embodiment of the present invention, a peak of a diffraction wavelength in a blue phase is not shifted to a longer wavelength side (i.e., light leakage from blue phase when no voltage is applied can be suppressed) and the upper limit of the temperature range where a blue phase is exhibited can be high.

Sample B1, Sample B2, and Comparative sample B1 each included in a liquid crystal element were subjected to polymer stabilization treatment. The polymer stabilization treatment was performed in such a manner that each of Sample B1, Sample B2, or Comparative sample B1 was set at a given constant temperature within the temperature range where a blue phase was exhibited, and ultraviolet light (peak wavelength of 365 nm, irradiance of 10 mW/cm²) irradiation was performed for 6 minutes. Through the polymer stabilization treatment, the polymerizable monomers in each of Sample B1, Sample B2, and Comparative sample B1 polymerized, so that Sample B1, Sample B2, and Comparative sample B1 each include an organic resin.

Next, in the liquid crystal elements each including Sample B1, Sample B2, and Comparative sample B1, which had been subjected to the polymer stabilization treatment, the spectra of the intensity of reflected light from the liquid crystal compositions were measured at room temperature with the microspectroscope.

In the liquid crystal element including Sample B1 and exhibiting a blue phase, the average peak of a diffraction wavelength was 425 nm. In the case of Sample B2, the average peak was 410 nm. In the case of Comparative sample B1, the average peak was 439 nm.

The phase transition temperature from a blue phase to an isotropic phase in the liquid crystal elements including Sample B1, Sample B2, and Comparative sample B1, which had been subjected to the polymer stabilization treatment, were measured with a polarizing microscope and a temperature controller.

The phase transition temperatures between a blue phase and an isotropic phase were 51.9° C. in Sample B1, 52.2° C. in Sample B2, and 48.6° C. in Comparative sample B1. Consequently, with the use of PPEP-7FFF or PPEP-9FFF, which is a liquid crystal material of one embodiment of the present invention, even in the case of a polymer stabilized blue phase, a peak of a diffraction wavelength is not shifted to a longer wavelength side and the upper limit of the temperature range where a blue phase is exhibited can be high.

This application is based on Japanese Patent Application serial no. 2013-159192 filed with Japan Patent Office on Jul. 31, 2013, the entire contents of which are hereby incorporated by reference.

What is claimed is:

1. A trifluorophenyl derivative represented by General Formula (G1): 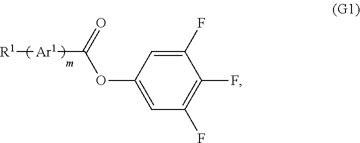
wherein:
Ar¹ represents an arylene group having 6 to 12 carbon atoms, a cycloalkylene group having 3 to 12 carbon atoms, or a cycloalkenylene group having 3 to 12 carbon atoms;
m represents 1 or 2; and
R¹ represents hydrogen, an alkyl group having 2 to 11 carbon atoms, or an alkoxy group having 2 to 11 carbon atoms.

2. A liquid crystal composition comprising:
the trifluorophenyl derivative according to claim 1; and
a chiral material.

3. A liquid crystal element comprising the liquid crystal composition according to claim 2.

4. A liquid crystal display device comprising the liquid crystal composition according to claim 2.

5. A trifluorophenyl derivative represented by General Formula (G2): 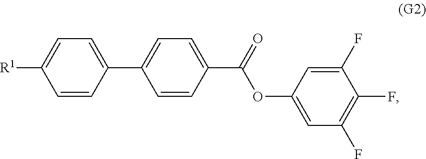
wherein R¹ represents hydrogen, an alkyl group having 2 to 11 carbon atoms, or an alkoxy group having 2 to 11 carbon atoms.

6. A liquid crystal composition comprising:
the trifluorophenyl derivative according to claim 5; and
a chiral material.

7. A liquid crystal element comprising the liquid crystal composition according to claim 6.

8. A liquid crystal display device comprising the liquid crystal composition according to claim 6.

9. A trifluorophenyl derivative represented by Structural Formula (103):

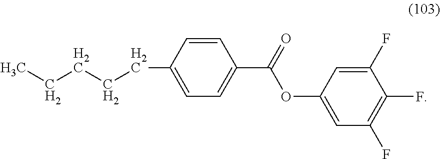 [SMILES: CCCCCc1ccc(C(=O)Oc2cc(F)c(F)c(F)c2)cc1]

10. A liquid crystal composition comprising:
the trifluorophenyl derivative according to claim 9; and
a chiral material.

11. A liquid crystal element comprising the liquid crystal composition according to claim 10.

12. A liquid crystal display device comprising the liquid crystal composition according to claim 10.

13. A trifluorophenyl derivative represented by Structural Formula (117):

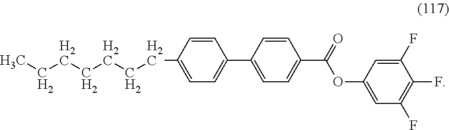 [SMILES: CCCCCCCc1ccc(-c2ccc(C(=O)Oc3cc(F)c(F)c(F)c3)cc2)cc1]

14. A liquid crystal composition comprising:
the trifluorophenyl derivative according to claim 13; and
a chiral material.

15. A liquid crystal element comprising the liquid crystal composition according to claim 14.

16. A liquid crystal display device comprising the liquid crystal composition according to claim 14.

17. A trifluorophenyl derivative represented by Structural Formula (119):

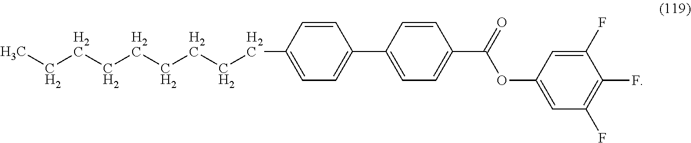 [SMILES: CCCCCCCCCc1ccc(-c2ccc(C(=O)Oc3cc(F)c(F)c(F)c3)cc2)cc1]

18. A liquid crystal composition comprising:
the trifluorophenyl derivative according to claim 17; and
a chiral material.

19. A liquid crystal element comprising the liquid crystal composition according to claim 18.

20. A liquid crystal display device comprising the liquid crystal composition according to claim 18.
